(19) United States
(12) Patent Application Publication
(10) Pub. No.: US 2022/0273726 A1
(43) Pub. Date: Sep. 1, 2022

(54) TREATMENT OF NEURONAL DISEASES

(71) Applicant: CENTER FOR EXCELLENCE IN BRAIN SCIENCE AND INTELLIGENCE TECHNOLOGY, CHINESE ACADEMY OF SCIENCES, Shanghai (CN)

(72) Inventors: Hui YANG, Shanghai (CN); Haibo ZHOU, Shanghai (CN)

(73) Assignee: CENTER FOR EXCELLENCE IN BRAIN SCIENCE AND INTELLIGENCE TECHNOLOGY, CHINESE ACADEMY OF SCIENCES, Shanghai (CN)

(21) Appl. No.: 17/627,052

(22) PCT Filed: Aug. 17, 2020
(86) PCT No.: PCT/CN2020/109655

§ 371 Date: Jan. 13, 2022

(30) Foreign Application Priority Data
Aug. 16, 2019 (CN) 201910760367.6
Oct. 30, 2019 (CN) 201911046435.9
Mar. 26, 2020 (CN) PCT/CN2020/081489
Jul. 28, 2020 (CN) 202010740568.2

(51) Int. Cl. A61K 35/30; A61P 25/28; A61K 38/17; A61K 48/00; C12N 15/86

(52) U.S. Cl. CPC A61K 35/30; A61P 25/28; A61K 38/17; A61K 48/005; C12N 15/86

(57) ABSTRACT

The invention described herein provides methods and compositions for treating certain neurodegenerative diseases, such as RGC loss-related degenerative disease and Parkinson's Disease, using in vivo conversion of glial cells to neurons by PTB and optionally nPTB knock down via CRISPR/Cas delivered by viral vectors (e.g., AAV vector).

### REFERENCE TO RELATED APPLICATIONS

This application claims priority to and the benefit of the filing date of patent application Nos. 201910760367.6, filed on Aug. 16, 2019, and patent application Nos. 201911046435.9, filed on Oct. 30, 2019 and patent application Nos. 202010740568.2 filed on Jul. 28, 2020, the entire contents of both applications are incorporated herein by reference.

### BACKGROUND OF THE INVENTION

Neurodegenerative diseases are devastating diseases associated with the progressive loss of neurons in various parts of the nervous system. On the other hand, regenerative medicine has great promise for treating neurodegenerative diseases that lead to cell (e.g., neuron) loss. One approach employs cell replacement, while another utilizes cellular trans-differentiation.

Trans-differentiation takes advantage of the existing cellular plasticity of endogenous cells to generate new cell types. One challenge for this approach, however, is to identify efficient strategies to convert certain target cells to a desired cell type (e.g., neurons), not only in culture but more importantly in their in vivo native contexts, particularly at a desired location (e.g., a tissue or organ type).

### SUMMARY OF THE INVENTION

In one aspect, the invention provides a method of generating a functional RGC (retinal ganglion cell) in an eye of a mammalian subject, comprising suppressing the expression or activity of PTB in a glial cell (e.g., a Muller glia cell) in the mature retina of the mammalian subject, and allowing said glial cell to reprogram into said RGC.

In certain embodiments, PTB expression or activity is suppressed by expressing in said glial cell a CRISPR/Cas effector protein and a guide RNA (gRNA) complementary to a PTB mRNA.

In certain embodiments, the Cas effector protein is selected from the group consisting of: Cas13d, CasRx, Cas13e, CRISPR/Cas9, Cpf1, Cas9, Cas13a, Cas13b, Cas13c, Cas13f and a combination thereof.

In certain embodiments, the gRNA targets nucleotides 4758-4787 and/or nucleotides 5381-5410 of the PTB coding sequence (e.g., GenBank 5725).

In certain embodiments, the CRISPR/Cas effector protein and/or the gRNA are encoded by an expression vector, and are optionally under the transcriptional control of a glial cell-specific promoter (such as GFAP promoter).

In certain embodiments, the expression vector comprises a viral vector.

In certain embodiments, the viral vector is selected from the group consisting of: an adeno-associated virus (AAV) vector, an adenovirus vector, a lentivirus vector, a retrovirus vector, a herpes virus, a SV40 vector, a poxvirus vector, and a combination thereof.

In certain embodiments, the viral vector is selected from the group consisting of: a lentivirus vector, an adenovirus vector, an adeno-associated virus (AAV) vector, and a combination thereof, preferably, the viral vector is an adeno-associated virus (AAV) vector or a lentivirus vector, more preferably, the viral vector is an adeno-associated virus (AAV) vector.

In certain embodiments, the Cas effector protein and two or more gRNA's, each specific for a different target region of the PTB mRNA, are encoded on one expression vector (such as AAV vector).

In certain embodiments, the AAV vector comprises AAV2, AAV2, or AAV9.

In certain embodiments, the glial cell is an MG cell.

In certain embodiments, the RGC (1) expresses Brn3a, Rbpms, Foxp2, Brn3c, and/or Parvalbumin; (2) is F-RGC, RGC subtype 3, or PV-RGC; (3) is integrated in existing retinal circuitry in said subject (e.g., capable of central projection to dLGN and partial visual restoration by relaying visual information to V1); and/or (4) is capable of receiving visual information characterized by ability to establish action potential upon light stimulation, synaptic connections (e.g., with existing functional dLGN neuron in the brain), biogenesis of pre-synaptic neurotransmitter, and/or post-synaptic response.

In certain embodiments, the method reprograms a plurality of glial cells in said mature retina, and wherein at least 10%, or at least 30% of said glial cells are converted to RGCs.

In certain embodiments, the subject is a human, or a non-human animal (such as mouse).

In certain embodiments, the subject is human, and wherein the method further comprises suppressing the expression or activity of nPTB in the glial cell, after an initial nPTB expression level increase to a high nPTB expression level following expression or activity of PTB is suppressed.

In certain embodiments, the high nPTB expression level is achieved about 3 days, 1 week, 10 days, 2 weeks, 3, weeks, or about 4 weeks after expression or activity of PTB is suppressed.

In certain embodiments, nPTB expression or activity is suppressed by expressing in said glial cell a CRISPR/Cas effector protein and a guide RNA (gRNA) complementary to an nPTB mRNA.

Another aspect of the invention provides a method of treating a neurological condition associated with degeneration of functional neurons in the mature retina of a subject in need thereof, comprising suppressing the expression or activity of PTB in a glial cell in the mature retina of the subject, and allowing said glial cell to reprogram into a functional neuron in the mature retina, thereby replenishing said degenerated functional neurons in said mature retina.

In certain embodiments, the neurological condition is selected from the group consisting of: glaucoma, age-related RGC loss, optic nerve injury, retinal ischemia, and Leber's hereditary optic neuropathy.

Another aspect of the invention provides a method of treating a neurological condition associated with degeneration of RGC neurons, comprising suppressing the expression or activity of PTB in a glial cell in the mature retina of a subject, and allowing said glial cell to reprogram into RGC neuron, thereby replenishing said degenerated RGC neurons in said mature retina.

Another aspect of the invention provides a method of generating a functional dopaminergic neuron in vivo, comprising suppressing the expression or activity of PTB in a glial cell in the striatum of a subject, and allowing said glial cell to reprogram into said dopaminergic neuron.

In certain embodiments, PTB expression or activity is suppressed by expressing in said glial cell a CRISPR/Cas effector protein and a guide RNA (gRNA) complementary to a PTB mRNA.

In certain embodiments, the Cas effector protein is selected from the group consisting of: Cas13d, CasRx, Cas13e, CRISPR/Cas9, Cpf1, Cas9, Cas13a, Cas13b, Cas13c, Cas13f and a combination thereof.

In certain embodiments, the CRISPR/Cas effector protein and/or said gRNA are encoded by an expression vector, and are optionally under the transcriptional control of a glial cell-specific promoter (such as GFAP promoter).

In certain embodiments, the Cas effector protein and two or more gRNA's, each specific for a different target region of the PTB mRNA, are encoded on one expression vector (such as AAV vector).

In certain embodiments, the AAV vector comprises AAV2, AAV2, or AAV9.

In certain embodiments, the glial cell is an astrocyte.

In certain embodiments, the dopaminergic neuron (1) expresses tyrosine hydroxylase (TH), dopamine transporter (DAT), vesicular monoamine transporter 2 (VMAT2), engrailed homeobox 1 (En1), FoxA2, and/or LEVI homeobox transcription factor 1 alpha (Lmx1a); (2) exhibits biogenesis of presynaptic neurotransmitter; (3) is integrated in existing neuronal circuitry in the brain of said subject; and/or (4) is characterized in its ability to establish action potential, synaptic connections, biogenesis of pre-synaptic neurotransmitter, and/or post-synaptic response.

In certain embodiments, the method reprograms a plurality of glial cells in said striatum, and wherein at least 10%, or at least 30% of said glial cells are converted to dopaminergic neurons.

In certain embodiments, the subject is a human, or a non-human animal (such as mouse).

In certain embodiments, the subject is human, and wherein the method further comprises suppressing the expression or activity of nPTB in the glial cell, after an initial nPTB expression level increase to a high nPTB expression level following expression or activity of PTB is suppressed.

In certain embodiments, the high nPTB expression level is achieved about 3 days, 1 week, 10 days, 2 weeks, 3, weeks, or about 4 weeks after expression or activity of PTB is suppressed.

In certain embodiments, nPTB expression or activity is suppressed by expressing in said glial cell a CRISPR/Cas effector protein and a guide RNA (gRNA) complementary to an nPTB mRNA.

Another aspect of the invention provides a method of treating a neurological condition associated with degeneration of functional neurons in the striatum of a subject in need thereof, comprising suppressing the expression or activity of PTB in a glial cell in the striatum of the subject, and allowing said glial cell to reprogram into a functional neuron in the striatum, thereby replenishing said degenerated functional neurons in said striatum. In certain embodiments, the neurological condition is selected from the group consisting of: Parkinson's disease; Alzheimer's disease; Huntington's disease; Schizophrenia; depression; drug addiction; movement disorder such as chorea, choreoathetosis, and dyskinesias; bipolar disorder; Autism spectrum disorder (ASD); and dysfunction.

Another aspect of the invention provides a method of treating a neurological condition associated with degeneration of dopaminergic neurons, comprising suppressing the expression or activity of PTB in a glial cell in the striatum of a subject, and allowing said glial cell to reprogram into dopaminergic neuron, thereby replenishing said degenerated dopaminergic neurons in said striatum.

Another aspect of the invention provides a method of restoring dopamine biogenesis in subject with a decreased amount of dopamine compared to a normal level, comprising suppressing the expression or activity of PTB in a glial cell in the striatum of a subject, and allowing said glial cell to reprogram into said dopaminergic neuron, thereby restoring at least 50% of said decreased amount of dopamine.

In certain embodiments, the glial cell is an astrocyte.

In certain embodiments, the neurological condition is Parkinson's disease.

In certain embodiments, a symptom is relieved in Parkinson's disease, wherein the symptom comprises tremor, stiffness, slowness, impaired balance, shuffling gait, postural instability, olfactory dysfunction, cognitive impairment, depression, sleep disorders, autonomic dysfunction, pain, and/or fatigue.

Another aspect of the invention provides a composition comprising a CRISPR/Cas effector protein or an expression vector encoding a CRISPR/Cas effector protein; and a guide RNA (gRNA) complementary to a PTB mRNA or an expression vector encoding a guide RNA (gRNA) complementary to a PTB mRNA.

In certain embodiments, the composition comprises a pharmaceutical composition.

In certain embodiments, the pharmaceutical composition is formulated for injection, inhalation, parenteral administration, intravenous administration, subcutaneous administration, intramuscular administration, intradermal administration, topical administration, or oral administration.

In certain embodiments, the expression vector encoding a CRISPR/Cas effector protein and the expression vector encoding a guide RNA (gRNA) complementary to a PTB mRNA are the same vector or different vectors.

Another aspect of the invention provide an injectable composition comprising an expression vector encoding a CRISPR/Cas construct configured to suppress expression or activity of PTB in a glial cell.

In certain embodiments, the expression vector comprises a viral vector.

In certain embodiments, the viral vector is selected from the group consisting of: an adeno-associated virus (AAV) vector, an adenovirus vector, a lentivirus vector, a retrovirus vector, a herpes virus, a SV40 vector, a poxvirus vector, and a combination thereof.

In certain embodiments, the viral vector is selected from the group consisting of: a lentivirus vector, an adenovirus vector, an adeno-associated virus (AAV) vector, and a combination thereof, preferably, the viral vector is an adeno-associated virus (AAV) vector or a lentivirus vector, more preferably, the viral vector is an adeno-associated virus (AAV) vector.

In certain embodiments, the glial cell is astrocyte, oligodendrocyte, ependymal cell, Schwan cell, NG2 cell, or satellite cell.

Another aspect of the invention provide an AAV vector, comprising:

(a) A coding sequence of a gene editing protein selected from the group consisting of CasRx, CRISPR/Cas9, Cpf1, Cas9, Cas13a, Cas13b, Cas13c, RNA targeted gene editing protein, and a combination thereof; and

(b) gRNA, which guides the gene editing protein to specifically bind to the DNA or RNA of the PTB gene.

In another preferred embodiment, the AAV vector further comprises a glial cell-specific promoter (for example, a GFAP promoter) for driving the expression of the gene editing protein.

It should be understood that any one embodiment of the invention described herein, including those described only in the examples or claims, or only in one aspects/sections below, can be combined with any other one or more embodiments of the invention, unless explicitly disclaimed or improper.

### BRIEF DESCRIPTION OF THE FIGURES

FIGS. 1A and 1B show knockdown efficiency of different combinations of gRNAs. The gRNA 5 and 6 showed the most potent knockdown efficiency in both N2a cells and astrocytes. Number above each bar indicates the number of repeats per group.

FIG. 2A is a schematic illustration of MG-to-RGC conversion via Ptbp1 knock-down in intact mature mice retinas. Vector I (AAV-GFAP-GFP-Cre) encodes Cre recombinase and GFP driven by the MG-specific promoter GFAP and Vector II (AAV-GFAP-CasRx-Ptbp1) encodes CasRx and gRNAs. To induce RGCs, retinas (Ai9 mice, 5 weeks old) were either injected with AAV-GFAP-CasRx-Ptbp1 or control vector AAV-GFAP-CasRx together with AAV-GFAP-GFP-Cre. Occurrence of conversion was examined around one month post-injection. ONL, outer nuclear layer; OPL, outer plexiform layer; INL, inner nuclear layer; IPL, inner plexiform layer; GCL, ganglion cell layer.

FIG. 2B is a schematic illustration of induction of RGCs from MG by knocking down Ptbp1 in the intact retinas of C57BL/6 mice. Vector 1 (GFAP-mCherry) encodes mCherry driven by the MG-specific promoter GFAP and Vector 2 (AAV-EFS-CasRx-Ptbp1) encodes gRNAs and CasRx under a ubiquitous promoter. To induce RGCs, retinas were either injected with AAV-GFAP-mCherry plus AAV-EFS-CasRx-Ptbp1, or AAV-GFAP-mCherry alone as a negative control. Occurrence of conversion was examined 2-3 weeks after injection.

FIG. 3A shows the number of tdTomato⁺ or tdTomato⁺ Brn3a⁺ cells in the GCL at one month after AAV injection. AAV-GFAP-GFP-Cre plus AAV-GFAP-CasRx, n=6 retinas; AAV-GFAP-GFP-Cre plus AAV-GFAP-CasRx-Ptbp1, n=7 retinas.

FIG. 3B shows the number of tdTomato⁺ or tdTomato⁺ Rbpms⁺ cells in the GCL at one month after AAV injection. AAV-GFAP-GFP-Cre plus AAV-GFAP-CasRx, n=6 retinas; AAV-GFAP-GFP-Cre plus AAV-GFAP-CasRx-Ptbp1, n=8 retinas. Data are presented as mean±SEM, *p<0.05, **p<0.01, ***p<0.001, unpaired t-test.

FIG. 4 is an outline of experimental design to show MG-to-RGC conversion in a mouse model of NMDA-induced retinal injury. Retinal injury was induced in Ai9 mice aged 4-8 weeks by intravitreal NMDA injection (200 mM, 1.5 μl). Two-three weeks after NMDA injection, AAVs were introduced by subretinal injection. Immunostaining and behavioral tests were performed one month after AAV injection.

FIG. 5A shows number of Brn3a⁺ or tdTomato⁺ or tdTomato⁺ Brn3a⁺ cells in the GCL. Uninjured retina, n=6 retinas; GFAP-CasRx plus GFAP-GFP-Cre, n=6 retinas; GFAP-CasRx-Ptbp1 plus GFAP-GFP-Cre, n=7 retinas.

FIG. 5B shows number of Rbpms⁺ or tdTomato⁺ or tdTomato⁺ Rbpms⁺ cells in the GCL. Uninjured retina, n=6 retinas; GFAP-CasRx plus GFAP-GFP-Cre, n=7 retinas; GFAP-CasRx-Ptbp1 plus GFAP-GFP-Cre, n=7 retinas.

FIG. 6 is a schematic illustration of the visual pathway. RGCs send their axons via the optic nerve that relay visual signals outside of the retina, to dLGN and SC in the brain.

FIG. 7 is a schematic illustration of VEP recording (C57BL/6 strain).

FIG. 8 shows response amplitude for wild type (WT, C57BL/6 strain, n=8 retinas), NMDA and AAV-GFAP-mCherry (n=12 retinas), NMDA, AAV-GFAP-mCherry and AAV-GFAP-CasRx (n=11 retinas), and NMDA, AAV-GFAP-mCherry and AAV-GFAP-CasRx-Ptbp1 (n=8 retinas). Each point represents a single mouse.

FIG. 9 shows design of the dark/light preference test. Note that both eyes were treated with NMDA and injected with the same AAVs two weeks later.

FIG. 10 shows percentage of time spent in dark chambers. WT (C57BL/6 strain), n=13 mice; NMDA and GFAP-mCherry, n=14 mice; NMDA, AAV-GFAP-mCherry and AAV-GFAP-CasRx, n=12 mice; NMDA, AAV-GFAP-mCherry and AAV-GFAP-CasRx-Ptbp1, n=12 mice. All values are presented as mean±SEM; unpaired t-test; *p<0.05, **p<0.01, ***p<0.001.

FIG. 11 shows the absolute number of tdTomato⁺ Brn3a⁺ and tdTomato⁺ Rbpms⁺ cells in the GCL. All values are presented as mean±SEM.

FIG. 12 is a schematic drawing showing the progressive projections of induced RGC axons over time.

FIG. 13 is a schematic illustration of the injection strategy. Vector I (AAV-GFAP-mCherry) encodes mCherry driven by the astrocyte-specific promoter GFAP. Vector II (AAV-GFAP-CasRx-Ptbp1) carries CasRx under GFAP promoter and gRNAs targeting Ptbp1. Striatum was either injected with AAV-GFAP-CasRx-Ptbp1 or control vector AAV-GFAP-CasRx together with AAV-GFAP-mCherry. Occurrence of conversion is evaluated around one-month post-injection. ST, striatum.

FIG. 14 shows quantification of Ptbp1 fluorescence intensity using ImageJ. a.u. stands for arbitrary unit.

FIG. 15 shows percentage of mCherry⁺ NeuN⁺ cells in mCherry⁺ cells (n=6 mice per group; t=−4.7, p<0.001).

FIG. 16 is an outline of the experiment showing conversion of striatal astrocytes into dopamine neurons in PD model mice.

FIG. 17 shows unilateral injection of 6-OHDA into the medial forebrain bundle. After 3 weeks, AAV-GFAP-CasRx-Ptbp1 plus AAV-GFAP-mCherry, AAV-GFAP-CasRx plus AAV-GFAP-mCherry or saline was injected into the ipsilateral (relative to the side of 6-OHDA infusion) striatum of mice infused with 6-OHDA. Immunostaining were performed around 1 month or 3 months after AAV injection.

FIG. 18 shows percentage of mCherry⁺ TH⁺ cells in mCherry⁺ cells. AAV-GFAP-CasRx, 1 month: n=5 mice; AAV-GFAP-CasRx-Ptbp1, 1 month: n=5 mice; AAV-GFAP-CasRx-Ptbp1, 3 months: n=3 mice.

FIG. 19 shows quantification of mCherry⁺ TH⁺ and mCherry⁺ DAT⁺ cells. n>=3 mice per group. Scale bar, 50 μm. Scale bar, 50 μm.

FIG. 20 shows percentage of mCherry⁺ TH⁺ cells in TH⁺ cells, n=5 mice per group. Scale bar, 50 μm.

FIG. 21 shows the percentage of mCherry⁺ TH⁺ cells in mCherry⁺ cells.

FIG. 22 shows percentage of mCherry⁺ DAT⁺ cells in mCherry⁺ cells. AAV-GFAP-CasRx, 1 month: n=5 mice; AAV-GFAP-CasRx-Ptbp1, 1 month: n=5 mice; AAV-GFAP-CasRx-Ptbp1, 3 months: n=3 mice.

FIG. 23 shows the absolute number of DAT+ cells in PD model mice.

FIG. 24 shows percentage of mCherry⁺ DAT⁺ TH⁺ cells in mCherry⁺ TH⁺ cells. AAV-GFAP-CasRx, 1 month: n=5 mice; AAV-GFAP-CasRx-Ptbp1, 1 month: n=5 mice; AAV-GFAP-CasRx-Ptbp1, 3 months: n=3 mice.

FIG. 25 shows percentage of mCherry⁺ FOXA2⁺ cells in mCherry⁺ cells. n=5 mice per group. FOXA2 is a dopamine neuron-marker. Scale bar, 30 μm.

FIG. 26 is an outline of the experiment showing astrocyte-to-neuron conversion alleviated motor dysfunctions in PD mice.

FIG. 27 shows net rotations (contralateral-ipsilateral) induced by apomorphine injection.

FIG. 28 shows comparison of the number of net rotations before and one month after AAV injection. Number above the dots indicates the number of mice per group. Paired t-test.

FIG. 29 shows net rotations (rotations/min) induced by apomorphine injection. Behavior was assessed at 1 month and 3 months for each mouse, n=3 mice per group. Two-way ANOVA followed by bonferroni's test.

FIG. 30 shows net rotations (ipsilateral-contralateral) induced by Amphetamine injection.

FIG. 31 shows the percentage of ipsilateral rotations relative to the total number of rotations (ipsilateral/total) after systemic injecting amphetamine.

FIG. 32 shows net rotations (rotations/min) induced by Amphetamine injection, n=3 mice per group. Two-way ANOVA followed by bonferroni's test. All values are presented as mean±SEM; unpaired t-test; *p<0.05, **p<0.01, ***p<0.001.

FIG. 33 shows the percentage of spontaneous ipsilateral touches, relative to the total number of touches.

FIG. 34 shows results of rotation test expressed as time (second) that mice remained on an accelerating rotarod before falling. Number above the bar indicates the number of mice per group. All values are presented as mean±SEM. One-way ANOVA followed by Tukey's test, *p<0.05, **p<0.01, ***p<0.001.

FIG. 35 shows the off-target detection scheme of in vitro Ptbp1 knockdown tool. The plasmids in the detection group are transfected into N2a cells (1), and the cells are collected after 48 hours, and the transfected positive cells are sorted by flow cytometry (2). After RNA extraction, transcriptome sequencing (3, RNA-seq) analysis is performed. In order to detect the off-target effect of different tools knocking down Ptbp1, the experiment sets up the Cas13e-sg (Ptbp1-target) experimental group and the Cas13e-sg (none-target) control group. At the same time, in order to compare with other gene editing tools, we set up Cas13d-sg (Ptbp1-target) and Cas13d-sg (none-target) control groups and shRNA (Ptbp1-target) and shRNA (none-target) control groups. The results show that the Cas13e-sg (Ptbp1-target) editing tool combination has a lower off-target effect.

FIG. 36 shows the detection scheme of transdifferentiation efficiency after Ptbp1 knockdown in vivo. This experiment uses the 6-OHDA-induced Parkinson's mouse model, by injecting 6-OHDA into the mouse MFB (Medial Forebrain Bundle) to damage dopamine neurons, and simulate the phenotype of Parkinson's disease. 21 days after the induction of the model, in the experimental group and the control group, AAV virus is injected in the mouse striatum respectively, and the AAV virus specifically labeled glial cells (GFAP promoter drives the expression of mCherry) is injected. After 28 or 90 days of virus injection, the transdifferentiation efficiency in vivo was verified by behavioral analysis and tissue staining analysis. The editing tools involved in this experiment all use AAV vectors. The experiment sets up experimental group of the Cas13e-sg (Ptbp1-target), as well as control group of Cas13e-sg (none-target), Cas13d-sg (Ptbp1-target) and Cas13d-sg (none-target). The above four sets of gene editing tools are all driven by the glial cell-specific GFAP promoter to express the Cas13e/d protein, and the U6 promoter is driven to express the corresponding sg. At the same time, the shRNA (Ptbp1-target) and shRNA (none-target) control groups are added, and the expression is driven by the U6 promoter. The results show that the Cas13e-sg (Ptbp1-target) editing tool combination can more effectively achieve the transdifferentiation of glial cells in vivo.

### DETAILED DESCRIPTION OF THE INVENTION

### 1. Overview

The invention described herein is partly based on the surprising discovery that a cell-programming agent that suppresses and/or inactivates PTB function, such as CRISPR/Cas effector protein coupled with a compatible guide RNA (gRNA) complementary to PTB mRNA, can be used to efficiently convert certain non-neuronal cells, such as the MG cells in mature retina, and glial cells (such as astrocytes) in striatum, into functional neurons (e.g., various RGC neurons and dopamine neurons), partly based on the local environment (e.g., region-dependent manner) into which such cell-programming agent is introduced.

This is in stark contrast to certain previous studies that concluded that certain other (i.e., non-Cas related) cell-programing agents targeting the same PTB transcript, such as RNAi reagents, expressed in the same non-neuronal cells, such as astrocytes in the striatum, do not seem to induce the astrocyte-to-neuronal conversion. In particular, in WO2019/200129, while it was shown that RNAi regents against PTB (serving as cell-programming agents) can apparently induce astrocyte-to-dopamine neuron conversion when such agents were introduced into the midbrain, the same agents failed to induce the same conversion in striatum. See FIG. 7G of WO2019/200129, and the corresponding description of the result: “[t]he near absence of astrocytes-derived TH-positive (dopamine) neurons in the striatum is striking, as this is the region innervated by the axons of nigral dopaminergic neurons.”

Thus one aspect of the invention provides a method of generating a functional dopaminergic neuron in vivo, comprising suppressing the expression or activity of PTB in a glial cell in the striatum of a subject, and allowing said glial cell to reprogram into said dopaminergic neuron.

In certain embodiments, PTB expression or activity is suppressed by expressing in the glial cell a CRISPR/Cas effector protein and a guide RNA (gRNA) complementary to a polynucleotide encoding PTB, such as PTB mRNA.

Numerous Cas effector proteins can be used in the methods and compositions of the invention, including those defined hereinabove, including but not limited to Cas13d, CasRx, Cas13e, or Cas13f. Such mRNA-targeting Cas effectors not only are more efficient and having less off-target effects than the RNAi reagents, but have added advantage (compared to other larger Cas effectors) of their shorter coding sequences which can be easily packaged into viral vectors with limited packaging capacity, such as AAV vectors. Further, targeting mRNA of PTB/nPTB, as opposed to the genes (DNA), is a safer clinical approach since it avoids permanent alteration of genomic DNA.

Thus in certain embodiments, the CRISPR/Cas effector protein and/or the gRNA are encoded by an AAV vector or a lentiviral vector. Though the Cas effector and gRNA do not need to be encoded by the same vector, they are conveniently encoded by the same vector to reduce the overall titer of viral vectors needed for in vivo therapeutic use, and to increase infection efficiency by avoiding the need for simultaneous co-infection by different types of viral vectors.

In certain embodiments, the encoded Cas effector and/or gRNA are under the transcriptional control of a glial cell-specific RNA Pol II promoter (such as the GFAP promoter). Cell-type specific expression of the Cas effector and gRNA enables tight control of the specific target non-neuronal cells to be converted to functional neurons for therapeutic use, while avoiding the undesirable outcome of unnecessarily converting other useful non-neuronal cell types to neurons, in undesirable locations, to disrupt the normal function of such non-neuronal cells.

In a related aspect, the invention also provides a method of generating a functional RGC (retinal ganglion cell) in an eye of a mammalian subject, comprising suppressing the expression or activity of PTB in a glial cell (e.g., a Muller glia cell) in the mature retina of the mammalian subject, and allowing said glial cell to reprogram into said RGC.

Such method can be used to at least partially restoring RGC function, in order to treat diseases or conditions resulting from or associated with loss of RGC neurons.

With the general aspects of the inventions described herein, specific aspects and embodiments of the invention are described further in the sections below, which should be viewed as a whole, and separate embodiments should be viewed as being capable of being combined with one another without restriction.

### 2. Definitions

“Astrocyte” generally refer to characteristic star-shaped glial cells in the brain and spinal cord, that is characterized by one or more of: star shape; expression of markers like glial fibrillary acidic protein (GFAP), aldehyde dehydrogenase 1 family member LI (ALDH1L1), excitatory amino acid transporter 1/glutamate aspartate transporter (EAAT1/GLAST), glutamine synthetase, S100 beta, or excitatory amino acid transporter 1/glutamate transporter 1 (EAAT2/GLT-1); participation of blood-brain barrier together with endothelial cells; transmitter uptake and release; regulation of ionic concentration in extracellular space; reaction to neuronal injury and participation in nervous system repair; and metabolic support of surrounding neurons.

In certain embodiments, an astrocyte refers to a non-neuronal cell in a nervous system that expresses glial fibrillary acidic protein (GFAP), Aldehyde Dehydrogenase 1 Family Member LI (ALDH1L1), or both.

In certain embodiments, an astrocyte refers to a non neuronal cell in a nervous system that expresses a glial fibrillary acidic protein (GFAP) promoter-driven transgene (e.g., red fluorescent protein (RFP), Cre recombinase).

A “BRN2 transcription factor” or “Brain-2 transcription factor,” also called “POU domain, class 3, transcription factor 2” (“POU3F2”) or “Oct-7,” can refer to a class III POU-domain transcription factor, having a DNA-binding POU domain that consists of an N-terminal POU-specific domain of about 75 amino acids and a C-terminal POU-homeo domain of about 60 amino acids, which are linked via a linker comprising a short a-helical fold, and which can be predominantly expressed in the central nervous system.

The term “cell-programming agent” generally refers to an agent that reprograms a differentiated non-neuronal cell to a neuronal cell, through inhibiting the expression and/or function of PTB and/or nPTB. In a specific embodiment, the cell-programming agent refers to a CRISPR/Cas effector protein (which may or may not include any variants, derivatives, functional equivalents or fragments thereof) with a guide RNA (gRNA) complementary to a PTB mRNA or to a nPTB mRNA, and can knock down the expression and/or activity of PTB or nPTB, to an extent sufficient to convert a non-neuronal cell to a neuronal cell, preferably in vivo at a local microenvironment where the converted neuron is expected to be functional. The cell-programming agent may also refer to a polynucleotide encoding such CRISPR/Cas effector protein as defined above and/or the guide RNA (gRNA). The polynucleotide may include an mRNA for the Cas effector as defined above. The polynucleotide may also include a DNA encoding the Cas effector as defined above and/or the gRNA complementary to PTB/nPTB mRNA. The DNA encoding the Cas effector as defined above and/or the gRNA may be part of a vector, including a viral vector (e.g., an AAV vector or a lentiviral vector, or any of the other viral vectors described hereinbelow). In the case of AAV, any AAV with tropism for glial cell or non-neuronal cell in the CNS and/or PNS can be used, including AAV1, AAV2, AAV3, AAV4, AAV5, AAV6, AAV7, AAV8, AAV9, AAV10, AAV11, AAV12, AAV13, AAV14, AAV15, AAV16, etc. Also in the case of AAV, any Cas effector as defined above can be used, so long as the coding sequence for the Cas effector is smaller than the packaging capacity of AAV, such as 4.7 kb, 4.5 kb, 4.0 kb, 3.5 kb, 3.0 kb, 2.5 kb, 2.0 kb, 1.5 kb or less. Exemplary Cas effector that may be used with the invention includes Cas13a, Cas13b, Cas13c, Cas13d, CasRx, Cas13e, Cas13f, Cpf1, Cas9, and functional equivalent or fragment thereof. In certain narrowest sense, the term “cell-programming agent” may be used interchangeably with the Cas effector with the gRNA, or polynucleotide (e.g., DNA or vector) encoding the same.

The Cas effector protein that can be used with the invention described herein include CRISPR-Cas Class 2 systems utilizing a single large Cas protein to degrade target nucleic acids (e.g., mRNA). Suitable Class 2 Cas effectors may include Type II Cas effectors such as Cas9 (e.g., Streptococcus pyogenes SpCas9 and S. thermophilus Cas9). The suitable Cas effector may also be Class 2, type V Cas proteins, including Cas12a (formerly known as Cpf1, such as Francisella novicida Cpf1 and Prevotella Cpf1), C2c1 and C2c3, which lack HNH nuclease, but have RuvC nuclease activity. Prevotella and Francisella lineages. Further suitable Cas effector proteins may include Class 2, type VI Cas proteins, including Cas13 (also known as C2c2), Cas13a, Cas13b, Cas13c, Cas13d/CasRx, CRISPR/Cas9, Cpf1, Cas13e and Cas13f, each is an RNA-guided RNase (i.e., these Cas proteins use their crRNA to recognize target RNA sequences, rather than target DNA sequences in Cas9 and Cas12a). Overall, the CRISPR/Cas13 systems can achieve higher RNA digestion efficiency compared to the traditional RNAi and CRISPRi technologies, while simultaneously exhibiting much less off-target cleavage compared to RNAi.

Thus in a specific embodiment, the cell-programming agent of the invention is or encodes a Cas effector protein that, together with its canonical gRNA, targets PTB or nPTB mRNA. In other embodiments, the Cas effector targets PTB or nPTB DNA.

The term “contacting” cells with a composition of the disclosure refers to placing the composition (e.g., compound, nucleic acid, viral vector etc.) in a location that will allow it to touch the cell in order to produce “contacted” cells. The contacting may be accomplished using any suitable method. For example, in one embodiment, contacting is by adding the compound to a culture of cells. Contacting may also be accomplished by injecting it or delivering the composition to a location within a body such that the composition “contacts” the cell type targeted.

The term “differentiation,” “differentiate,” or “converting,” or “inducing differentiation” are used interchangeably to refer to changing the default cell type (genotype and/or phenotype) to a non-default cell type (genotype and/or phenotype). Thus “inducing differentiation in an astrocyte cell” refers to inducing the cell to change its morphology from that of an astrocyte to that of a neuronal cell type (i.e., change in gene expression as determined by genetic analysis such as a microarray) and/or phenotype (i.e. change in expression of a protein).

The term “glial cell” can generally refer to a type of supportive cell in the central nervous system (e.g., brain and spinal cord) and the peripheral nervous system.

In some embodiments, glial cells do not conduct electrical impulses or exhibit action potential. In some embodiments, glial cells do not transmit information with each other, or with neurons via synaptic connection or electrical signals. In a nervous system or in an in vitro culture system, glial cells can surround neurons and provide support for and insulation between neurons. Non-limiting examples of glial cells include oligodendrocytes, astrocytes, ependymal cells, Schwann cells, microglia, and satellite cells.

A “microRNA” or “miRNA” refers to a non-coding nucleic acid (RNA) sequence that binds to at least partially complementary nucleic acid sequence (mRNAs) and negatively regulates the expression of the target mRNA at the post-transcriptional level. A microRNA is typically processed from a “precursor” miRNA having a double-stranded, hairpin loop structure to a “mature” form. Typically, a mature microRNA sequence is about 19-25 nucleotides in length.

“miR-9” is a short non-coding RNA gene involved in gene regulation and highly conserved from Drosophila and mouse to human. The mature ˜21nt miRNAs are processed from hairpin precursor sequences by the Dicer enzyme. miR-9 can be one of the most highly expressed microRNAs in developing and adult vertebrate brain. Key transcriptional regulators such as FoxGl, Hesl or Tlx, can be direct targets of miR-9, placing it at the core of the gene network controlling the neuronal progenitor state.

The term “neuron” or “neuronal cell” as used herein can have the ordinary meaning one skilled in the art would appreciate. In some embodiments, neuron can refer to an electrically excitable cell that can receive, process, and transmit information through electrical signals (e.g., membrane potential discharges) and chemical signals (e.g., synaptic transmission of neurotransmitters). As one skilled in the art would appreciate, the chemical signals (e.g., based on release and recognition of neurotransmitters) transduced between neurons can occur via specialized connections called synapses.

The term “mature neuron” can refer to a differentiated neuron. In some embodiments, a neuron is the to be a mature neuron if it expresses one or more markers of mature neurons, e.g., microtubule-associated protein 2 (MAP2) and Neuronal Nuclei (NeuN), neuron specific enolase (NSE), 160 kDa neurofilament medium, 200 kDa neurofilament heavy, postsynaptic density protein 95 (PDS-95), Synapsin I, Synaptophysin, glutamate decarboxylase 67 (GAD67), glutamate decarboxylase 67 (GAD65), parvalbumin, dopamine- and cAMP-regulated neuronal phosphoprotein 32 (DARPP32), vesicular glutamate transporter 1 (vGLUT1), vesicular glutamate transporter 2 (vGLUT2), acetylcholine, and tyrosine hydroxylase (TH).

The term “functional neuron” can refer to a neuron that is able to send or receive information through chemical or electrical signals. In some embodiments, a functional neuron exhibits one or more functional properties of a mature neuron that exists in a normal nervous system, including, but not limited to: excitability (e.g., ability to exhibit action potential, e.g., a rapid rise and subsequent fall in voltage or membrane potential across a cellular membrane), forming synaptic connections with other neurons, pre-synaptic neurotransmitter release, and post-synaptic response (e.g., excitatory postsynaptic current or inhibitory postsynaptic current). In some embodiments, a functional neuron is characterized in its expression of one or more markers of functional neurons, including, but not limited to, synapsin, synaptophysin, glutamate decarboxylase 67 (GAD67), glutamate decarboxylase 67 (GAD65), parvalbumin, dopamine- and cAMP-regulated neuronal phosphoprotein 32 (DARPP32), vesicular glutamate transporter 1 (vGLUT1), vesicular glutamate transporter 2 (vGLUT2), acetylcholine, tyrosine hydroxylase (TH), dopamine, vesicular GABA transporter (VGAT), and gamma-aminobutyric acid (GABA).

The term “non-neuronal cell” can refer to any type of cell that is not a neuron. An exemplary non neuronal cell is a cell that is of a cellular lineage other than a neuronal lineage (e.g., a hematopoietic lineage). In some embodiments, a non-neuronal cell is a cell of neuronal lineage but not a neuron, for example, a glial cell. In some embodiments, a non-neuronal cell is somatic cell that is not neuron, such as, but not limited to, glial cell, adult primary fibroblast, embryonic fibroblast, epithelial cell, melanocyte, keratinocyte, adipocyte, blood cell, bone marrow stromal cell, Langerhans cell, muscle cell, rectal cell, or chondrocyte. In some embodiments, a non-neuronal cell is from a non-neuronal cell line, such as, but not limited to, glioblastoma cell line, Hela cell line, NT2 cell line, ARPE19 cell line, or N2A cell line.

“Cell lineage” or “lineage” can denote the developmental history of a tissue or organ from the fertilized embryo.

“Neuronal lineage” can refer to the developmental history from a neural stem cell to a mature neuron, including the various stages along this process (as known as neurogenesis), such as, but not limited to, neural stem cells (neuroepithelial cells, radial glial cells), neural progenitors (e.g., intermediate neuronal precursors), neurons, astrocytes, oligodendrocytes, and microglia.

The terms “nucleic acid” and “polynucleotide” as used interchangeably herein can refer to deoxyribonucleotides or ribonucleotides and polymers thereof in either single- or double-stranded form. The term can encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, locked nucleic acids (LNAs), and peptide-nucleic acids (PNAs).

“Oligodendrocyte” can refer to a type of glial call that can create myelin sheath that surrounds a neuronal axon to provide support and insulation to axons in the central nervous system. Oligodendrocyte can also be characterized in their expression of PDGF receptor alpha (PDGFR-α), SOXIO, neural/glial antigen 2 (NG2), Olig 1, 2, and 3, oligodendrocyte specific protein (OSP), Myelin basic protein (MBP), or myelin oligodendrocyte glycoprotein (MOG).

“Polypyrimidine tract binding protein” or “PTB” and its homolog neural PTB (nPTB) are both ubiquitous RNA-binding proteins. PTB can also be called polypyrimidine tract-binding protein 1, and in humans is encoded by the PTBP1 gene. PTBP1 gene belongs to the subfamily of ubiquitously expressed heterogeneous nuclear ribonucleoproteins (hnRNPs).

The hnRNPs are RNA-binding proteins and they complex with heterogeneous nuclear RNA (hnRNA). These proteins are associated with pre-mRNAs in the nucleus and appear to influence pre-mRNA processing and other aspects of mRNA metabolism and transport. PTB can have four repeats of quasi-RNA recognition motif (RRM) domains that bind RNAs. Consistent with its widespread expression, PTB can contribute to the repression of a large number of alternative splicing events. PTB can recognize short RNA motifs, such as UCUU and UCUCU, located within a pyrimidine-rich context and often associated with the polypyrimidine tract upstream of the 3′ splice site of both constitutive and alternative exons.

In some cases, binding site for PTB can also include exonic sequences and sequences in introns downstream of regulated exons.

In most alternative splicing systems regulated by PTB, repression can be achieved through the interaction of PTB with multiple PTB binding sites surrounding the alternative exon. In some cases, repression can involve a single PTB binding site. Splicing repression by PTB can occur by a direct competition between PTB and U2AF65, which in turn can preclude the assembly of the U2 snRNP on the branch point. In some cases, splicing repression by PTB can involve PTB binding sites located on both sides of alternative exons, and can result from cooperative interactions between PTB molecules that would loop out the RNA, thereby making the splice sites inaccessible to the splicing machinery. Splicing repression by PTB can also involve multimerization of PTB from a high-affinity binding site that can create a repressive wave that covers the alternative exon and prevents its recognition.

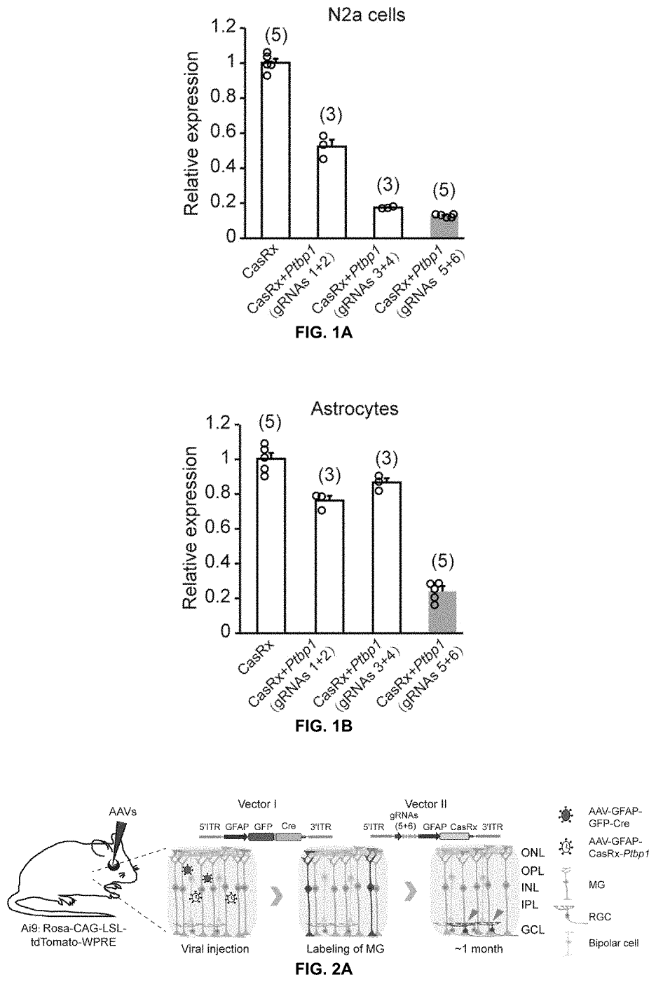

PTB can be widely expressed in non-neuronal cells, while nPTB can be restricted to neurons. PTB and nPTB can undergo a programmed switch during neuronal differentiation. For example, as illustrated in FIG. 1, during neuronal differentiation, PTB is gradually down-regulated at the neuronal induction stage, coincidentally or consequentially, nPTB level is gradually up-regulated to a peak level. Later, when the neuronal differentiation enters into neuronal maturation stage, nPTB level experiences reduction after its initial rise and then returns to a relatively low level as compared to the its peak level during neuronal differentiation, when the cell develops into a mature neuron.

The sequences of PTB and nPTB are known (see, e.g., Romanelli et al. (2005) Gene 356:11-8; Robinson et al., PLoS One. (2008) 3(3):e1801. doi:10.1371/journal.pone.0001801; Makeyev et al., Mol. Cell (2007) 27(3):435-448); thus, one of skill in the art can design and construct gRNA molecules and the like to modulate, e.g., to decrease or inhibit, the expression of PTB/nPTB; to practice the methods of the invention.

The terms “protein,” “peptide,” and “polypeptide” are used interchangeably, and can refer to an amino acid polymer or a set of two or more interacting or bound amino acid polymers.

The term “promoter” can refer to an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. Promoters include constitutive and inducible promoters. A “constitutive” promoter is a promoter that can be active under most environmental and developmental conditions.

An “inducible” promoter is a promoter that can be active under environmental or developmental regulation.

The term “operably linked” can refer to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The term “reprogramming” or “trans-differentiation” can refer to the generation of a cell of a certain lineage (e.g., a neuronal cell) from a different type of cell (e.g., a fibroblast cell) without an intermediate process of de differentiating the cell into a cell exhibiting pluripotent stem cell characteristics.

“Pluripotent” can refer to the ability of a cell to form all lineages of the body or soma (i.e., the embryo proper). Exemplary “pluripotent stem cells” can include embryonic stem cells and induced pluripotent stem cells.

The terms “subject” and “patient” as used interchangeably can refer to, except where indicated, mammals such as humans and non-human primates, as well as rabbits, rats, mice, goats, pigs, and other mammalian species. The term does not necessarily indicate that the subject has been diagnosed with a particular disease, but instead can refer to an individual under medical supervision.

For example, mammalian species that benefit from the disclosed methods and composition include, but are not limited to, primates, such as apes, chimpanzees, orangutans, humans, monkeys; domesticated animals (e.g., pets) such as dogs, cats, guinea pigs, hamsters, Vietnamese pot-bellied pigs, rabbits, and ferrets; domesticated farm animals such as cows, buffalo bison, horses, donkey, swine, sheep, and goats; exotic animals typically found in zoos such as bear, lions, tigers, panthers, elephants, hippopotamus, rhinoceros, giraffes antelopes, sloth, gazelles, zebras, wildebeests, prairie dogs, koala bears, kangaroo opossums, raccoons, pandas, hyena, seals, sea lions, elephant seals, otters, porpoises dolphins, and whales.

A “vector” is a nucleic acid that can be capable of transporting another nucleic acid into a cell. A vector can be capable of directing expression of a protein or proteins encoded by one or more genes, or a microRNA encoded by a polynucleotide, carried by the vector when it is present in the appropriate environment.

A “viral vector” is a viral-derived nucleic acid that can be capable of transporting another nucleic acid into a cell. A viral vector can be capable of directing expression of a protein or proteins encoded by one or more genes, or a microRNA encoded by a polynucleotide, carried by the vector when it is present in the appropriate environment. Examples of viral vectors include, but are not limited to, retroviral, adenoviral, lentiviral and adeno-associated viral vectors.

As used herein and in the appended claims, the singular forms “a,” “an,” and “the” include plural referents unless the context clearly dictates otherwise. Thus, for example, reference to “an inhibitor” includes a plurality of inhibitors and reference to “the agent” includes reference to one or more agents and equivalents thereof known to those skilled in the art, and so forth.

Unless defined otherwise, all technical and scientific terms used herein have the same meaning as commonly understood to one of ordinary skill in the art to which this disclosure belongs. Although any methods and reagents similar or equivalent to those described herein can be used in the practice of the disclosed methods and compositions, the exemplary methods and materials are now described.

All publications, patents, and patent applications mentioned herein are incorporated herein by reference in full for the purpose of describing and disclosing the methodologies, which are described in the publications, which might be used in connection with the description herein, as if each individual publication, patent, or patent application was specifically and individually indicated to be incorporated by reference. With respect to any term that is presented in one or more publications that is similar to, or identical with, a term that has been expressly defined in this disclosure, the definition of the term as expressly provided herein will control in all respects.

Terms such as “comprise,” “comprises,” “comprising,” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

### 3. Methods and Compositions

In one aspect, the invention described herein provides compositions and methods to convert or differentiate non-neuronal mammalian cells such as glial cells (e.g., MG cells or astrocytes) into functional neurons (e.g., RGC neurons or dopamine neurons in striatum) by knocking down Polypyrimidine Tract Binding protein (PTB).

Specifically, one aspect of the invention provides methods of reprogramming a non-neuronal cell to a mature neuron. An exemplary method comprises: providing a non-neuronal cell, and contacting the non-neuronal cell with a composition comprising a cell-programming agent (such as CRISPR/Cas effector with guide RNA (gRNA) or polynucleotide encoding the same) that suppresses expression and/or activity of PTB and/or nPTB in the non-neuronal cell, thereby reprogramming the non neuronal cell to a mature neuron. The methods and compositions not only convert cells in vitro but also directly in vivo in brain (such as in striatum).

According to some embodiments of the disclosure, a single cell-programming agent (e.g., Cas/gRNA) that suppresses the expression and/or activity of PTB/nPTB in a human non-neuronal cell (e.g., MG cell in mature retina or astrocyte in striatum) can directly convert the non-neuronal cell into a mature neuron (e.g., RGC neuron or dopamine neuron, respectively), e.g., when the human non-neuronal cell expresses miR-9 or Brn2 at a level that is higher than that expressed in a human adult fibroblast. In some embodiments, the direct conversion of a non-neuronal cell into a neuron by a single cell-programming agent (e.g., Cas/gRNA) can mean that the conversion of the non-neuronal cell into the neuron requires no other intervention than contacting with the single cell-programming agent.

An exemplary method comprises: providing a human non neuronal cell that expresses miR-9 or Brn2 at a level that is higher than that expressed in a human adult fibroblast; and contacting the human non-neuronal cell with a composition comprising a cell-programming agent (e.g., Cas/gRNA) that suppresses expression and/or activity of PTB/nPTB in the human non-neuronal cell, thereby reprogramming the human non-neuronal cell to a mature neuron.

In some embodiments, human glial cell can express miR-9 or Brn2 at a level that is higher than that expressed in a human adult fibroblast. In another embodiment, the disclosure provides a method of reprogramming a human glial cell to a mature neuron. An exemplary method comprises: providing the human glial cell to be reprogrammed; and contacting the human glial cell with a composition comprising a cell-programming agent that suppresses the expression or activity of PTB and/or nPTB (e.g., Cas with PTB/nPTB-targeting gRNA, or polynucleotide encoding the same) in the human glial cell for at least 1 day, thereby reprogramming the human glial cell to a mature neuron.

In another embodiment, the disclosure provides a method of reprogramming an astrocyte to a mature neuron. An exemplary method comprises: providing the astrocyte to be reprogrammed; and contacting the astrocyte with a composition comprising a cell programming agent (e.g., Cas with PTB/nPTB-targeting gRNA or polynucleotide encoding the same) that suppresses the expression or activity of PTB in the astrocyte for at least 1 day, thereby reprogramming the astrocyte to a mature neuron such as a dopamine neuron. In some embodiments, a single cell programming agent (e.g., Cas with PTB-targeting gRNA) that suppresses the expression or activity of PTB in an astrocyte can directly convert the astrocyte into a neuron such as a dopamine neuron. In some embodiments, the astrocyte is in striatum.

In another embodiment, the invention provides a method of reprogramming an MG cell (e.g., one in mature retina) to an RGC neuron. An exemplary method comprises: providing the MG cell to be reprogrammed; and contacting the MG cell with a composition comprising a cell programming agent (e.g., Cas with PTB/nPTB-targeting gRNA or polynucleotide encoding the same) that suppresses the expression or activity of PTB and/or nPTB in the MG cell for at least 1 day, thereby reprogramming the MG cell to an RGC neuron. In some embodiments, a single cell programming agent (e.g., Cas with PTB-targeting gRNA) that suppresses the expression or activity of PTB in an MG cell can directly convert the MG cell into an RGC neuron. In some embodiments, the MG cell is in mature retina.

In some embodiments, the methods provided herein comprise administering the cell-programming agent (e.g., a Cas effector protein and a coding sequence for an anti-PTB/nPTB gRNA) to a target non-neuronal cell (e.g., a glial cell or astrocyte in the striatum, or a MG cell in the mature retina).

In certain embodiments, the cell-programming agent (e.g., a Cas effector protein and a coding sequence for an anti-PTB/nPTB gRNA) is delivered as a viral vector, such as an AAV vector (e.g., AAV1, AAV2, or AAV9). In certain embodiments, the viral vector contains a glial cell or astrocyte specific promoter (such as GFAP) that only transcribes the Cas effector and gRNA in a cell-type specific manner.

According to the disclosure, in some cases, PTB reduction can induce a number of key neuronal differentiation factors. For example, without wishing to be bound to a certain theory, PTB and nPTB can be involved in two separate but intertwined loops, separately, that can be important in neuronal differentiation. PTB can suppress a neuronal induction loop in which the microRNA miR-124 can inhibit the transcriptional repressor RE1-Silencing Transcription factor (REST), which in turn can block the induction of miR-124 and many neuronal-specific genes (loop I). During a normal neuronal differentiation process, PTB can be gradually down-regulated, and the PTB down-regulation can thus induce the expression of nPTB, which is part of a second loop for neuronal maturation that includes the transcription activator Brn2 and miR-9 (loop II). In loop II, nPTB can inhibit Brn2 and consequentially can inhibit miR-9, and miR-9 in turn can inhibit nPTB.

According to some embodiments, the expression level of miR-9 or Brn2 in a non-neuronal cell can affect the conversion of the non-neuronal cell into a mature neuron by a cell-programming agent that suppresses the expression or activity of PTB in the non-neuronal cell. For example, a human adult fibroblast cell can have a low expression level of miR-9 and Brn2. In some embodiments, a single agent that suppresses the expression or activity of PTB in a human adult fibroblast cell can induce the human adult fibroblast cell to differentiate into a neuron-like cell, e.g., expression of Tuj1 protein, but not into a mature neuron, e.g., expression of NeuN protein or other markers of a mature neuron.

Without wishing to be bound by a particular theory, the subject method and composition in some embodiments are particularly effective in creating a reinforcing feedback loop in molecular changes that direct the conversion of a non-neuronal cell into a neuron. Without wishing to be bound by a particular theory, when PTB expression or activity is initially downregulated by an exogenous anti-PTB agent, REST level can be downregulated, which can in turn lead to upregulation of miR-124 level.

Without wishing to be bound by a particular theory, in some cases, because miR-124 can target and inhibit the expression of PTB, the upregulated miR-124 can thus reinforce the inhibition of PTB in the cell; such a positive reinforcing effect can be long-lasting, even though in some cases, the anti-PTB agent, e.g., an antisense oligonucleotide against PTB, may be present and active merely temporarily in the cell.

According to some embodiments of the disclosure, a single cell-programming agent (e.g., Cas with PTB/nPTB-targeting gRNA or polynucleotide encoding the same) that suppresses the expression or activity of PTB/nPTB in a human non-neuronal cell can directly convert the non-neuronal cell into a mature neuron, optionally when the human non neuronal cell expresses miR-9 or Brn2 at a level that is higher than that expressed in a human adult fibroblast.

An exemplary human non-neuronal cell that can be used in the method provided herein expresses miR-9 or Brn2 at a level that is at least two times higher than that expressed in a human adult fibroblast. In some embodiments, the human non-neuronal cell expresses miR-9 or Brn2 at a level that is at least about 1.2 times, at least about 1.5 times, at least about 1.6 times, at least about 1.8 times, at least about 2 times, at least about 2.5 times, at least about 3 times, at least about 3.5 times, at least about 4 times, at least about 4.5 times, at least about 5 times, at least about 5.5 times, at least about 6 times, at least about 6.5 times, at least about 7 times, at least about 7.5 times, at least about 8 times, at least about 8.5 times, at least about 9 times, at least about 9.5 times, at least about 10 times, at least about 11 times, at least about 12 times, at least about 15 times, at least about 20 times, or at least about 50 times higher than that expressed in a human adult fibroblast.

In some embodiments, a single cell-programming agent that suppresses the expression or activity of PTB/nPTB (e.g., Cas with PTB/nPTB-targeting gRNA) in a human non neuronal cell can directly convert the non-neuronal cell into a mature neuron, when the human non-neuronal cell expresses both miR-9 and Brn2 at a level that is higher than that expressed in a human adult fibroblast.

An exemplary human non-neuronal cell that can be used in the method as provided herein express both miR-9 and Brn2 at a level that is at least two times higher than that expressed in a human adult fibroblast. In some embodiments, the human non neuronal cell expresses both miR-9 and Brn2 at a level that is at least about 1.2 times, at least about 1.5 times, at least about 1.6 times, at least about 1.8 times, at least about 2 times, at least about 2.5 times, at least about 3 times, at least about 3.5 times, at least about 4 times, at least about 4.5 times, at least about 5 times, at least about 5.5 times, at least about 6 times, at least about 6.5 times, at least about 7 times, at least about 7.5 times, at least about 8 times, at least about 8.5 times, at least about 9 times, at least about 9.5 times, at least about 10 times, at least about 11 times, at least about 12 times, at least about 15 times, at least about 20 times, or at least about 50 times higher than that expressed in a human adult fibroblast.

In some embodiments, a single cell-programming agent (e.g., Cas with PTB/nPTB-targeting gRNA) that suppresses the expression or activity of PTB/nPTB in a human non-neuronal cell can directly convert the non-neuronal cell into a mature neuron, when the human non-neuronal cell expresses endogenous miR-9 or endogenous Brn2 at a level that is higher than that expressed in a human adult fibroblast. In some embodiments, no exogenous miR-9 is introduced into the human non-neuronal cell. In some embodiments, no exogenous Brn2 is introduced into the human non-neuronal cell.

In some embodiments, the expression level of miR-9 or Brn2 in a non-neuronal cell can be assessed by any technique one skilled in the art would appreciate. For example, the expression level of miR-9 in a cell can be measured by reverse transcription (RT)-polymerase chain reaction (PCR), miRNA array, RNA sequencing (RNA-seq), and multiplex miRNA assays. Expression level of miR-9 can also be assayed by in situ methods like in situ hybridization. Expression level of Brn2 as a protein can be assayed by conventional techniques, like Western blot, enzyme-linked immunosorbent assay (ELISA), and immunostaining, or by other techniques, such as, but not limited to, protein microarray, and spectrometry methods (e.g., high-performance liquid chromatography (HPLC) and liquid chromatography-mass spectrometry (LC/MS)). In some embodiments, information on the expression level of miR-9 in a cell or a certain type of tissue/cells can be obtained by referring to publicly available databases for microRNAs, such as, but not limited to, Human MiRNA Expression Database (HMED), miRGator 3.0, miRmine, and PhenomiR. In some embodiments, information on the expression level of miR-9 in a cell or a certain type of tissue/cells can be obtained by referring to publicly available databases for protein expression, including, but not limited to, The Human Protein Atlas, GeMDBJ Proteomics, Human Proteinpedia, and Kahn Dynamic Proteomics Database.

According to certain embodiments, an exemplary method comprises providing a human non-neuronal cell to be reprogrammed; and contacting the human non-neuronal cell with a composition comprising a single cell-programming agent (e.g., Cas with PTB/nPTB-targeting gRNA) that yields a decrease in expression or activity of PTB in the human non neuronal cell, and a decrease of expression or activity of nPTB after the expression or activity of PTB is decreased. In some embodiments, the cell-programming agent can lead to a sequential event as to the expression or activity levels of PTB and nPTB in a certain type of non-neuronal cell, e.g., human non-neuronal cell, e.g., human glial cell. In some embodiments, the direct effect of contacting with the cell-programming agent (e.g., Cas with PTB-targeting gRNA) is a decrease of expression or activity of PTB in the non-neuronal cell. In some embodiments, in the non-neuronal cell, the decrease of expression or activity of PTB in the non-neuronal cell accompanies an initial increase of nPTB expression level in the non-neuronal cell. In some embodiments, an initial nPTB expression level increases to a high nPTB expression level as expression or activity of PTB is suppressed. In some embodiments, following the initial increase, nPTB expression decreases from the high nPTB expression level to a low nPTB expression level. In some embodiments, the low nPTB expression level is still higher than the initial nPTB expression level after expression or activity of PTB is suppressed. In some embodiments, the nPTB expression level decreases after the initial increase spontaneously without external intervention other than the cell-programming agent that suppresses the expression or activity of PTB. Without being bound to a certain theory, the subsequent decrease of nPTB expression level in the non-neuronal cell after PTB expression or activity is decreased by the cell-programming agent can be correlated with the direct conversion of the non neuronal cell to a mature neuron by the cell-programming agent. According to some embodiments, a single cell-programming agent (e.g., Cas with PTB-targeting gRNA) that suppresses the expression or activity of PTB does not induce the sequential event as described above in a human adult fibroblast cell, e.g., nPTB can experience the initial rise in expression level, but no subsequent decrease to a certain low level. In some embodiments, in a human astrocyte, a single cell-programming agent (e.g., Cas with PTB-targeting gRNA) that suppresses the expression or activity of PTB in the human astrocyte leads to immediate decrease in expression or activity of PTB, an initial increase in expression level of nPTB, and a subsequent decrease in expression level of nPTB. In some embodiments, a single cell-programming agent (e.g., Cas with PTB-targeting gRNA) that suppresses the expression or activity of PTB directly converts a human astrocyte to a mature neuron. In some embodiments, the expression level of miR-9 or Brn2 in the non-neuronal cell can be correlated with whether or not nPTB expression level in the non-neuronal decreases after the initial increases following PTB expression or activity is suppressed by a cell-programming agent. For instance, in human astrocyte, where miR-9 or Brn2 is expressed at a higher level than a human adult fibroblast, nPTB expression level in the non-neuronal decreases after the initial increases following PTB expression or activity is suppressed by a cell-programming agent, while in human adult fibroblast, as described above, in some cases, the subsequent decrease in nPTB expression level may not happen.

According to some embodiments, an exemplary non-neuronal cell that can be reprogrammed into a mature neuron in the method provided herein can include a glial cell, such as, but not limited, astrocyte, oligodendrocyte, ependymal cell, Schwan cell, NG2 cells, and satellite cell. In some embodiments, a glial cell can be a human glial cell, for instance, human astrocyte. In some embodiments, a glial cell can be a mouse glial cell. In some embodiments, a glial cell can be a glial cell from any other mammals, such as, but not limited to, non-human primate animals, pigs, dogs, donkeys, horses, rats, rabbits, and camels.

In some embodiments, a glial cell that can be used in the method as provided herein is a glial cell isolated from a brain. In some embodiments, a glial cell is a glial cell in a cell culture, for instance, divided from a parental glial cell. In some embodiments, a glial cell as provided herein is a glial cell differentiated from a different type of cell under external induction, for instance, differentiated in vitro from a neuronal stem cell in a culture medium containing differentiation factors, or differentiated from an induced pluripotent stem cell. In some other embodiments, a glial cell is a glial cell in a nervous system, for example, a MG cell in the mature retina, or an astrocyte residing in a brain region, such as in the striatum.

In some embodiments, an astrocyte that can be used in the method as provided herein is a glial cell that is of a star-shape in brain or spinal cord. In some embodiments, an astrocyte expresses one or more of well-recognized astrocyte markers, including, but not limited to, glial fibrillary acidic protein (GFAP) and aldehyde dehydrogenase 1 family member LI (ALDH1L1), excitatory amino acid transporter 1/glutamate aspartate transporter (EAAT1/GLAST), glutamine synthetase, S100 beta, or excitatory amino acid transporter 1/glutamate transporter 1 (EAAT2/GLT-1). In some embodiments, an astrocyte expresses glial fibrillary acidic protein (GFAP), Aldehyde Dehydrogenase 1 Family Member LI (ALDH1L1), or both. In certain embodiments, an astrocyte is a non-neuronal cell in a nervous system that expresses a glial fibrillary acidic protein (GFAP) promoter-driven transgene (e.g., red fluorescent protein (RFP), Cre recombinase). In some embodiments, an astrocyte as described herein is not immunopositive for neuronal markers, e.g., Tuj1, NSE, NeuN, GAD67, VGluT1, or TH. In some embodiments, an astrocyte as described herein is not immunopositive for oligodendrocyte markers, e.g., Oligodendrocyte Transcription Factor 2, OLIG2. In some embodiments, an astrocyte as described herein is not immunopositive for microglia markers, e.g., transmembrane protein 119 (TMEM119), CD45, ionized calcium binding adapter molecule 1 (Iba1), CD68, CD40, F4/80, or CD11 Antigen-Like Family Member B (CD11b). In some embodiments, an astrocyte as described herein is not immunopositive for NG2 cell markers (e.g., Neural/glial antigen 2, NG2). In some embodiments, an astrocyte as described herein is not immunopositive for neural progenitor markers, e.g., Nestin, CXCR4, Musashi, Notch-1, SRY-Box 1 (SOX1), SRY-Box 2 (SOX2), stage-specific embryonic antigen 1 (SSEA-1, also called CD15), or Vimentin. In some embodiments, an astrocyte as described herein is not immunopositive for pluripotency markers, e.g., NANOG, octamer-binding transcription factor 4 (Oct-4), SOX2, Kruppel Like Factor 4 (KLF4), SSEA-1, or stage-specific embryonic antigen 4 (SSEA-4). In some embodiments, an astrocyte as described herein is not immunopositive for fibroblast markers (e.g, Fibronectin).

Astrocytes can include different types or classifications. The methods of the invention are applicable to different types of astrocytes. Non-limiting example of different types of astrocytes include type 1 astrocyte, which can be Ran2⁺, GFAP⁺, fibroblast growth factor receptor 3 positive (FGFR3⁺), and A2B5. Type 1 astrocytes can arise from the tripotential glial restricted precursor cells (GRP). Type 1 astrocytes may not arise from the bipotential 02A/0PC (oligodendrocyte, type 2 astrocyte precursor) cells. Another non limiting example includes type 2 astrocyte, which can be A2B5⁺, GFAP⁺, FGFR3⁻, and Ran2. Type 2 astrocytes can develop in vitro from either tripotential GRP or from bipotential 02A cells or in vivo when these progenitor cells are transplanted into lesion sites. Astrocytes that can be used in the method provided herein can be further classified based their anatomic phenotypes, for instance, protoplasmic astrocytes that can be found in grey matter and have many branching processes whose end-feet envelop synapses; fibrous astrocyte that can be found in white matter and can have long thin unbranched processes whose end-feet envelop nodes of Ranvier. Astrocytes that can be used in the methods provided herein can also include GluT type and GluR type. GluT type astrocytes can express glutamate transporters (EAAT1/SLC1A3 and EAAT2/SLC1A2) and respond to synaptic release of glutamate by transporter currents, while GluR type astrocytes can express glutamate receptors (mostly mGluR and AMPA type) and respond to synaptic release of glutamate by channel-mediated currents and IP3-dependent Ca²⁺ transients.

### 3. Cell-Programming Agent

As provided herein, a cell-programming agent (e.g., Cas with PTB-targeting gRNA or polynucleotide encoding the same) suppresses expression or activity of PTB by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% of the endogenous or native level.

As provided herein, cell-programming agent (e.g., Cas with nPTB-targeting gRNA or polynucleotide encoding the same) suppresses expression or activity of nPTB by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, or about 100% of the endogenous or native level.

In some embodiments, a cell-programming agent as provided herein (e.g., Cas with PTB/nPTB-targeting gRNA or polynucleotide encoding the same) directly suppress the expression level of PTB/nPTB, e.g., suppressing the transcription, translation, or protein stability of PTB and/or nPTB.

In some embodiments, a cell programming agent as provided herein (e.g., Cas with PTB/nPTB-targeting gRNA or polynucleotide encoding the same) directly effects on the expression or activity of PTB/nPTB, without affecting other cellular signaling pathway.

As provided herein, a cell-programming agent that suppresses the expression or activity of PTB/nPTB is a CRISPR/Cas family effector protein, such as CRISPR/Cas9, Cpf1, Cas9, Cas13a, Cas13b, Cas13c, Cas13d, CasRx, Cas13e, or Cas13f, or a functional domain thereof. In certain embodiments, the CRISPR/Cas family effector protein is encoded by an ORF (from start codon to stop codon) of 4.5 kb or less, 4 kb or less, 3.5 kb or less, 3 kb or less, 2.5 kb or less, or 2.1 kb or less, or 1.5 kb or less.

As provided herein, contacting the non-neuronal cell with a cell-programming agent can be performed in any appropriate manner, depending on the type of non-neuronal cell to be reprogrammed, the environment in which the non-neuronal cell resides, and the desired cell reprogramming outcome.

In these configurations, non-viral transfection methods or viral transduction methods are utilized to introduce the cell-programming agent. Non-viral transfection can refer to all cell transfection methods that are not mediated through a virus. Non-limiting examples of non-viral transfection include electroporation, microinjection, calcium phosphate precipitation, transfection with cationic polymers, such as DEAE-dextran followed by polyethylene glycol, transfection with dendrimers, liposome mediated transfection (“lipofection”), microprojectile bombardment (“gene gun”), fugene, direct sonic loading, cell squeezing, optical transfection, protoplast fusion, impalefection, magnetofection, nucleofection, and any combination thereof.

In some embodiments, the methods provided herein utilize viral vectors as appropriate medium for delivering the cell programming agent to the non-neuronal cell. As provided herein, viral vector methods can include the use of either DNA or RNA viral vectors. Examples of appropriate viral vectors can include adenoviral, lentiviral, adeno-associated viral (AAV), poliovirus, herpes simplex virus (HSV), or murine Maloney-based viral vectors.

In some embodiments, the vector is an AAV vector. In some embodiments, a cell-programming agent is administered in the form of AAV vector. In some embodiments, a cell-programming agent is administered in the form of lentiviral vector. For example, a cell-programming agent can be delivered to a non-neuronal cell using a lentivirus or adenovirus associated virus (AAV) to express a Cas effector protein with gRNA against PTB/nPTB.

According to some embodiments of the disclosure, methods provided herein comprise suppressing the expression or activity of PTB/nPTB in a non-neuronal cell (e.g., a glia cell or astrocyte) via a cell-programming agent of a sufficient amount for reprogramming the non-neuronal cell to a mature neuron. The sufficient amount of cell-programming agent can be determined empirically as one skilled in the art would readily appreciate. In some embodiments, the amount of cell-programming agent can be determined by any type of assay that examines the activity of the cell-programming agent in the non-neuronal cell.

For example, when the cell-programming agent is configured to suppress the expression of PTB/nPTB in the non-neuronal cell, the sufficient amount of the cell-programming agent can be determined by assessing the expression level of PTB/nPTB in an exemplary non neuronal cell after administration of the agent, e.g., by Western blot. In some embodiments, functional assays are utilized for assessing the activity of PTB/nPTB after delivery of the cell programming agent to an exemplary non-neuronal cell. In some embodiments, other functional assays, such as, immunostaining for neuronal markers, electrical recording for neuronal functional properties, that examine downstream neuronal properties are used to determine a sufficient amount of cell-programming agent.

In some embodiments, the cell-programming agent is delivered in the form of a viral vector. A viral vector can comprises one or more copies of expression sequence coding for a cell-programming agent, e.g., a Cas effector protein with one or more copies of coding sequence for gRNA against PTB/nPTB, such as, 1, 2, 3, 4, 5, 6, 7, 8, 9, 10 copies.

A viral vector can be tittered to any appropriate amount for administration, as one skilled in the art will be able to determine. For example, the titer as determined by PCR, RT-PCR, or other methods can be at least about 10⁵ viral particles/mL, at least about 10⁶ particles/mL, at least about 10⁷ particles/mL, at least about 10⁸ particles/mL, at least about 10⁹ particles/mL, at least about 10¹⁰ particles/mL, at least about 10¹¹ particles/mL, at least about 10¹² particles/mL, at least about 10¹³ particles/mL, at least about 10¹⁴ particles/mL, or at least about 10¹⁵ particles/mL.

In some embodiments, the titer of viral vector to be administered is at least about 10¹⁰ viral particles/mL, at least about 10¹¹ viral particles/mL, at least about 10¹² viral particles/mL, at least about 10¹³ viral particles/mL, or at least about 10¹⁴ viral particles/mL.

### 4. Dosing and Treatment Regimens

Methods provided herein can comprise suppressing the expression or activity of PTB/nPTB in a non-neuronal cell for a certain period of time sufficient for reprogramming the non-neuronal cell to a mature neuron.

In some embodiments, exemplary methods comprise contacting the non-neuronal cell with a cell-programming agent that suppresses the expression or activity of PTB/nPTB in the non neuronal cell for at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 3 weeks, at least 4 weeks, at least 5 weeks, at least 2 months, at least 3 months, at least 4 months, or at least 5 months, thereby reprogramming the non-neuronal cell to a mature neuron.

In certain embodiments, suppression of PTB and nPTB expression or activity is sequential. For example, the expression or activity of PTB is first suppressed for, e.g., any one of the above-mentioned time period, before the expression or activity of nPTB is suppressed.

In certain embodiments, suppression of PTB and nPTB expression or activity is concurrent.

In some embodiments, exemplary methods comprise contacting the non-neuronal cell with a cell-programming agent that suppresses the expression or activity of PTB in the non-neuronal cell for about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 3 weeks, about 4 weeks, about 5 weeks, about 2 months, about 3 months, about 4 months, or about 5 months, before contacting the non-neuronal cell with a cell-programming agent that suppresses the expression or activity of nPTB in the non-neuronal cell, thereby reprogramming the non-neuronal cell to a mature neuron.

In some configurations, the methods provided herein comprise administering the cell-programming agent for only once, e.g., adding the cell-programming agent to a cell culture comprising the non-neuronal cell, or delivering the cell programming agent to a brain region comprising the non-neuronal cell (e.g., striatum), for only once, and the cell-programming agent can remain active as suppressing expression or activity of PTB/nTPB in the non neuronal cell for a desirable amount of time, e.g., for at least 1 day, at least 2 days, at least 4 days, or at last 10 days. For instance, when the cell-programming agent comprises an AAV vector expressing a Cas effector and a coding sequence for an anti-PTB gRNA, the design of the AAV vector can enable it to remain transcriptionally active for an extended period of time.

In some embodiments, the methods provided herein comprise administering the cell-programming agent for more than once, e.g., for at least 2 times, at least 3 times, at least 4 times, at least 5 times, at least 6 times, at least 7 times, at least 8 times, at least 9 times, at least 10 times, at least 12 times, at least 15 times, at least 20 times or more.

### 5. In Vitro Medium and Culture

As provided herein, a method can comprise reprogramming a non-neuronal cell to a neuron in vitro under appropriate culture conditions. One of ordinary skills in the art will appreciate that appropriate cell culture conditions can be chosen for promoting neuronal growth. In some embodiments, various factors can be provided in the culture medium for maintaining the survival of the non-neuronal cells, the cells undergoing reprogramming, and the reprogrammed neurons. Any known culture medium capable of supporting cell growth can be used and optimized for desirable outcomes. Culture medium can include HEM, DMEM, RPMI, F-12, or the like. Culture medium can contain supplements which can be important for cellular metabolism such as glutamine or other amino acids, vitamins, minerals or useful proteins such as transferrin and the like. Medium can also contain antibiotics to prevent contamination with yeast, bacteria and fungi such as penicillin, streptomycin, gentamicin and the like. In some cases, the medium can contain serum derived from bovine, equine, chicken and the like. An exemplary culture medium for astrocyte as a starting cell can include DMEM/F12, FBS, penicillin/streptomycin, B27, epidermal growth factor (EGF), and fibroblast growth factor 2 (FGF2). In some cases, a neuron differentiation medium is used during the reprogramming and/or maintaining the reprogramed neurons. In some cases, a neuron differentiation medium comprises an inhibitor of ALK5 (TGF type I receptor kinase), such as SB431542, A-77-01, ALK5 inhibitor II, RepSox, SB525334, GW788388, SD-208, LY215729, or LY364947.

In some cases, a neuron differentiation medium comprises an inhibitor of GSK3b (glycogen synthase kinase 3 beta), such as CHIR99021, IM-12, TWS119, BIO, 3F8, AR-A014418, AT9283, or 2-Thio (3-iodobenzyl)-5-(1-pyridyl)[1,3,4]-oxadiazole. In some cases, a neuron differentiation medium comprises an activator of PKA (protein kinase A), such as dibutyryl-cAMP (cyclic adenosine monophosphate), 8-bromo-cAMP, 8-CPT-cAMP, taxol, belinostat, or Sp-cAMPs. An exemplary neuronal differentiation medium includes a N3/basal medium, containing DMEM/F12, Neurobasal, insulin, transferring, sodium lenite, progesterone, putrescine, supplemented with B27, FBS, ChIR99021, SB431542 and db-cAMP, and/or neurotrophic factors like BDNF, GDNF, NT3 and CNTF.

### 6. Markers

According to some embodiments of the present disclosure, the methods provided herein comprise reprogramming a plurality of non-neuronal cells into mature neurons at a high efficiency.

In some embodiments, the methods comprise reprogramming MG cells or astrocytes into mature neurons, and at least 60% of the MG cells/astrocytes are converted to mature neurons that are Tuj1 positive.

In some embodiments, at least 40% of the MG cells/astrocytes are converted to mature neurons that are Map2 positive. In some embodiments, at least about 20%, at least about 25%, at least about 30%, at least about 35%, at least about 38%, at least about 40%, at least about 42%, at least about 44%, at least about 46%, at least about 48%, at least about 50%, at least about 52%, at least about 54%, at least about 56%, at least about 58%, at least about 60%, at least about 62%, at least about 64%, at least about 66%, at least about 68%, at least about 70%, at least about 72%, at least about 74%, at least about 76%, at least about 78%, at least about 80%, at least about 82%, at least about 84%, at least about 86%, at least about 88%, at least about 90%, at least about 92%, at least about 94%, at least about 96%, at least about 98%, at least about 99%, or 100% of the MG cells/astrocytes are converted to mature neurons that are positive for Tuj1 or Map2.

In some embodiments, the methods comprise reprogramming human astrocytes into mature neurons, and at least 40%, at least 60%, or at least 80% of the human astrocytes are converted to mature neurons that are Tuj1 positive. In some embodiments, at least 20%, at least 40% or at least 60% of the human astrocytes are converted to mature neurons that are Map2 positive. In some embodiments, at least about 20%, at least about 25%, at least about 30%, at least about 35%, at least about 38%, at least about 40%, at least about 42%, at least about 44%, at least about 46%, at least about 48%, at least about 50%, at least about 52%, at least about 54%, at least about 56%, at least about 58%, at least about 60%, at least about 62%, at least about 64%, at least about 66%, at least about 68%, at least about 70%, at least about 72%, at least about 74%, at least about 76%, at least about 78%, at least about 80%, at least about 82%, at least about 84%, at least about 86%, at least about 88%, at least about 90%, at least about 92%, at least about 94%, at least about 96%, at least about 98%, at least about 99%, or about 100% of the human astrocytes are converted to mature neurons that are positive for Tuj1 or Map2.

In some embodiments, the methods as provided herein comprise reprogramming a plurality of non-neuronal cells, e.g., human non-neuronal cells, e.g., human glial cells, or astrocytes, and at least about 20%, at least about 25%, at least about 30%, at least about 35%, at least about 38%, at least about 40%, at least about 42%, at least about 44%, at least about 46%, at least about 48%, at least about 50%, at least about 52%, at least about 54%, at least about 56%, at least about 58%, at least about 60%, at least about 62%, at least about 64%, at least about 66%, at least about 68%, at least about 70%, at least about 72%, at least about 74%, at least about 76%, at least about 78%, at least about 80%, at least about 82%, at least about 84%, at least about 86%, at least about 88%, at least about 90%, at least about 92%, at least about 94%, at least about 96%, at least about 98%, or at least about 99% of the non-neuronal cells, e.g., human non-neuronal cells, e.g., human glial cells, or astrocytes are reprogrammed to mature neurons. In some embodiments, the methods as provided herein reprogram about 20%, about 25%, about 30%, about 35%, about 38%, about 40%, about 42%, about 44%, about 46%, about 48%, about 50%, about 52%, about 54%, about 56%, about 58%, about 60%, about 62%, about 64%, about 66%, about 68%, about 70%, about 72%, about 74%, about 76%, about 78%, about 80%, about 82%, about 84%, about 86%, about 88%, about 90%, about 92% about 94%, about 96% about 98% about 99%, or about 100% of the non-neuronal cells, e.g., human non-neuronal cells, e.g., human glial cells, MG cells, or astrocytes are reprogrammed to mature neurons.

In some embodiments, a mature neuron is characterized by its expression of one or more neuronal markers selected from the group consisting of NeuN (neuronal nuclei antigen), Map2 (microtubule-associated protein 2), NSE (neuron specific enolase), 160 kDa neurofilament medium, 200 kDa neurofilament heavy, PDS-95 (postsynaptic density protein 95), Synapsin I, Synaptophysin, GAD67 (glutamate decarboxylase 67), GAD65 (glutamate decarboxylase 67), parvalbumin, DARPP32 (dopamine- and cAMP-regulated neuronal phosphoprotein 32), vGLUT1 (vesicular glutamate transporter 1), vGLUT2 (vesicular glutamate transporter 1), acetylcholine, vesicular GABA transporter (VGAT), and gamma-aminobutyric acid (GABA), and TH (tyrosine hydroxylase). In some embodiments, at least 40% of the non-neuronal cells, e.g., human non-neuronal cells, e.g., human glial cells, or astrocytes are reprogrammed to mature neurons.

As one of ordinary skills in the art would readily appreciate, the expression of all those markers above can be assessed by any common techniques. For examples, immunostaining using antibodies against specific cell type markers as described herein can reveal whether or not the cell of interest expresses the corresponding cell type marker. Immunostaining under certain conditions can also uncover the subcellular distribution of the cell type marker, which can also be important for determining the developmental stage of the cell of interest. For instance, expression of Map2 can be found in various neurites (e.g., dendrites) in a postmitotic mature neuron, but absent in axon of the neuron. Expression of voltage-gated sodium channels (e.g., a subunits Navi.1-1.9) and b subunits) can be another example, they can be clustered in a mature neuron at axon initial segment, where action potential can be initiated, and Node of Ranvier. In some embodiments, other techniques, such as, but not limited to, flow cytometry, mass spectrometry, in situ hybridization, RT-PCR, and microarray, can also be used for assessing expression of specific cell type markers as described herein.

### 7. Conversion Efficiency

Certain aspects of the present disclosure provide methods that comprise reprogramming a plurality of non-neuronal cells, and at least about 20%, at least about 25%, at least about 30%, at least about 35%, at least about 38%, at least about 40%, at least about 42%, at least about 44%, at least about 46%, at least about 48%, at least about 50%, at least about 52%, at least about 54%, at least about 56%, at least about 58%, at least about 60%, at least about 62%, at least about 64%, at least about 66%, at least about 68%, at least about 70%, at least about 72%, at least about 74%, at least about 76%, at least about 78%, at least about 80%, at least about 82%, at least about 84%, at least about 86%, at least about 88%, at least about 90%, at least about 92%, at least about 94%, at least about 96%, at least about 98%, or at least about 99% of the non-neuronal cells, e.g., human non-neuronal cells, e.g., human glial cells, MG cells, or astrocytes are reprogrammed to functional neurons.

In some embodiments, the methods provided herein reprogram at least 20% of the non-neuronal cells, e.g., human non neuronal cells, e.g., human glial cells, MG cells, or astrocytes are reprogrammed to functional neurons. In some embodiments, the methods provided herein reprogram about 20%, about 25%, about 30%, about 35%, about 38%, about 40%, about 42%, about 44%, about 46%, about 48%, about 50%, about 52%, about 54%, about 56%, about 58%, about 60%, about 62%, about 64%, about 66%, about 68%, about 70%, about 72%, about 74%, about 76%, about 78%, about 80%, about 82%, about 84%, about 86%, about 88%, about 90% about 92% about 94%, about 96%, about 98%, about 99%, or about 100% of the non-neuronal cells, e.g., human non-neuronal cells, e.g., human glial cells, MG cells, or astrocytes are reprogrammed to functional neurons.

### 8. Functional Assessment

In some embodiments, functional neurons are characterized in their ability to form neuronal network, to send and receive neuronal signals, or both. In some embodiments, functional neurons fire action potential. In some embodiments, functional neurons establish synaptic connections with other neurons. For instance, a functional neuron can be a postsynaptic neuron in a synapse, e.g., having its dendritic termini, e.g., dendritic spines, forming postsynaptic compartments in synapses with another neuron. For instance, a functional neuron can be a presynaptic neuron in a synapse, e.g., having axonal terminal forming presynaptic terminal in synapses with another neuron.

Synapses a functional neuron can form with another neuron can include, but not limited to, axoaxonic, axodendritic, and axosomatic. Synapses a functional neuron can form with another neuron can be excitatory (e.g., glutamatergic), inhibitory (e.g., GABAergic), modulatory, or any combination thereof. In some embodiments, synapses a functional neuron forms with another neuron is glutamatergic, GABAergic, cholinergic, adrenergic, dopaminergic, or any other appropriate type. As a presynaptic neuron, a function neuron can release neurotransmitter such as, but not limited to, glutamate, GABA, acetylcholine, aspartate, D-serine, glycine, nitric oxide (NO), carbon monoxide (CO), hydrogen sulfide (H2S), dopamine, norepinephrine (also known as noradrenaline), epinephrine (adrenaline), histamine, serotonin, phenethylamine, N-methylphenethylamine, tyramine, 3-iodothyronamine, octopamine, tryptamine, somatostatin, substance P, opioid peptides, adenosine triphosphate (ATP), adenosine, and anandamide. As a postsynaptic neuron, a functional neuron can elicit postsynaptic response to a neurotransmitter released by a presynaptic neuron into the synaptic cleft. The postsynaptic response a functional neuron as generated in the method provided herein can be either excitatory, inhibitory, or any combination thereof, depending on the type of neurotransmitter receptor the functional neuron expresses. In some embodiments, the functional neuron expresses ionic neurotransmitter receptors, e.g., ionic glutamate receptors and ionic GABA receptors. Ionic glutamate receptors can include, but not limited to, a-amino-3-hydroxy-5-methyl-4-isoxazolepropionic acid (AMPA)-type glutamate receptors (e.g., GluA1/GRIA1; GluA2/GRIA2; GluA3/GRIA3; GluA4/GRIA4), delta receptors (e.g., GluD1/GRID1; GluD2/GRID2), kainate receptors (e.g., GluK1/GRIK1; GluK2/GRIK2; GluK3/GRIK3; GluK4/GRIK4; GluK5/GRIK5) and iV-methyl-D-aspartate (NMDA) receptors (e.g., GluN1/GRIN1; GluN2A/GRIN2A; GluN2B/GRIN2B; GluN2C/GRIN2C; GluN2D/GRIN2D; GluN3A/GRIN3A; GluN3B/GRIN3B). Ionic GABA receptors can include, but not limited to, GABAA receptor. In some embodiments, the functional neuron expresses metabolic neurotransmitter receptors, e.g., metabolic glutamate receptors (e.g., mGluRi, mGluRs, mGluR, mGluR, mGluRi, mGluRe, mGluR, mGluRs), and metabolic GABA receptors (e.g., GABAB receptor). In some embodiments, the functional neuron expresses a type of dopamine receptor, either D1-like family dopamine receptor, e.g., D1 and D5 receptor (DIR and D5R), or D2-like family dopamine receptor, e.g., D2, D3, and D4 receptors (D2R, D3R, and D4R). In some embodiments, a functional neuron as provided herein forms electrical synapse with another neuron (e.g., gap junction). In some embodiments, a function neuron as provided herein forms either chemical or electrical synapse (s) with itself, as known as autapse.

The characteristics of a function neuron can be assessed by common techniques available to one skilled in the art. For example, the electrical properties of a functional neuron, such as, firing of action potential and postsynaptic response to neurotransmitter release can be examined by techniques such as patch clamp recording (e.g., current clamp and voltage clamp recordings), intracellular recording, and extracellular recordings (e.g., tetrode recording, single-wire recording, and filed potential recording). Specific properties of a functional neuron (e.g., expression of ion channels and resting membrane potential) can also be examined by patch clamp recording, where different variants of patch clamp recording can be applied for different purposes, such as cell-attached patch, inside-out patch, outside-out patch, whole-cell recording, perforated patch, loose patch. Assessment of postsynaptic response by electrical methods can be coupled with either electrical stimulation of presynaptic neurons, application of neurotransmitters or receptor agonists or antagonists. In some cases, AMPA-type glutamate receptor-mediated postsynaptic current can be assessed by AMPA receptor agonists, e.g., AMPA, or antagonists, e.g., 2, 3-dihydroxy-6-nitro-7-sulfamoyl-benzoquinoxaline (NBQX) or 6-cyano-7-nitroquinoxaline-2,3-dione (CNQX). In some cases, NMDA-type glutamate receptor-mediated postsynaptic current can be assessed by NMDA receptor agonists, e.g., NMDA and glycine, or antagonists, e.g., AP5 and ketamine.

In some embodiments, functional neurons are examined by techniques other than electrical approaches. For example, recent development of various fluorescent dyes or genetically encoded fluorescent proteins and imaging techniques can be utilized for monitoring electrical signals conveyed or transmitted by a functional neuron. In this context, calcium-dependent fluorescent dyes (e.g., calcium indicators), such as, but not limited to, fura-2, indo-1, fluo-3, fluo-4, and Calcium Green-1, and calcium-dependent fluorescent proteins, such as, but not limited to, Cameleons, FIP-CBSM, Pericams, GCaMP, TN-L15, TN-humTnC, TN-XL, TN-XXL, and Twitch's, can be used to trace calcium influx and efflux as an indicator of neuronal membrane potential. Alternatively or additionally, voltage-sensitive dyes that can change their spectral properties in response to voltage changes can also be used for monitoring neuronal activities.

Neurotransmitter release can be an important aspect of a functional neuron. The methods provided herein can comprise reprogram a non-neuronal cell to a functional neuron that releases a certain type of neurotransmitter. In some embodiments, the functional neuron releases neurotransmitter such as, but not limited to, glutamate, GABA, acetylcholine, aspartate, D-serine, glycine, nitric oxide (NO), carbon monoxide (CO), hydrogen sulfide (H2S), dopamine, norepinephrine (also known as noradrenaline), epinephrine (adrenaline), histamine, serotonin, phenethylamine, N-methylphenethylamine, tyramine, 3-iodothyronamine, octopamine, tryptamine, somatostatin, substance P, opioid peptides, adenosine triphosphate (ATP), adenosine, and anandamide.

In some embodiments, the functional neuron releases dopamine as a major neurotransmitter. In some embodiments, the functional neuron releases more than one type of neurotransmitter. In some embodiments, the functional neuron releases neurotransmitter in response to an action potential. In some embodiments, the functional neuron releases neurotransmitter in response to graded electrical potential (e.g., membrane potential changes that do not exceed a threshold for eliciting an action potential). In some embodiments, the functional neuron exhibits neurotransmitter release at a basal level (e.g., spontaneous neurotransmitter release). Neurotransmitter release as described herein from a functional neuron can be assessed by various techniques that are available to one of ordinary skills in the art. In some embodiments, imaging approaches can be used for characterizing a functional neuron's neurotransmitter release, for instance, by imaging a genetically encoded fluorescent fusion molecule comprising a vesicular protein, one can monitor the process of synaptic vesicles being fused to presynaptic membrane.

Alternatively or additionally, other methods can be applied to directly monitor the level of a specific neurotransmitter. For example, HPLC probe can be used to measure the amount of dopamine in a culture dish or a brain region where a functional neuron projects its axon to. The level of dopamine as detected by HPLC can indicate the presynaptic activity of a functional neuron. In some embodiments, such assessment can be coupled with stimulation of the functional neuron, in order to change its membrane potential, e.g., to make it elicit action potential.

In an aspect, the present disclosure provides a method of generating a functional neuron in vivo. An exemplary method comprises administering to a region in the nervous system, e.g., mature retina or a region in the brain or spinal cord (e.g., striatum), of a subject a composition comprising a cell programming agent (e.g., a Cas effector protein and a gRNA targeting/complementing to PTB and/or nPTB, or polynucleotide encoding the same) in a non-neuronal cell (e.g., a glial cell or astrocyte) in the region in nervous system, and allowing the non-neuronal cell to reprogram into the functional neuron. In some embodiments, the cell-programming agent suppresses the expression or activity of PTB and/or nPTB. In some embodiments, the cell-programming agent does not comprise NeuroD1 protein, or an expression construct coding for NeuroD1.

### 9. Administration Route

According to some embodiments of the present disclosure, the methods provided herein comprise direct administration of a cell-programming agent (e.g., a Cas effector protein and a gRNA targeting/complementing to PTB and/or nPTB or polynucleotide encoding the same) into a region in the nervous system (e.g., mature retina or a region in the brain or spinal cord (e.g., striatum)) of a subject. In some embodiments, the cell programming agent (e.g., a Cas effector protein and a gRNA targeting/complementing to PTB and/or nPTB or polynucleotide encoding the same) is delivered locally to a region in the nervous system (e.g., mature retina or a region in the brain or spinal cord (e.g., striatum)). In one embodiment, a composition comprising a cell programming agent, such as a viral vector (e.g, AAV vector), is administered to the subject or organism by stereotaxic or convection enhanced delivery to a brain region (e.g., striatum).

Using stereotaxic positioning system, one skilled in the art would be able to locate a specific brain region (e.g., striatum) that is to be administered with the composition comprising the cell-programming agent. Such methods and devices can be readily used for the delivery of the composition as provided herein to a subject or organism. In another embodiment, a composition as provided herein is delivered systemically to a subject or to a region in nervous system, e.g., brain (e.g., striatum) or spinal cord, of a subject, e.g., delivered to cerebrospinal fluid or cerebral ventricles, and the composition comprises one or more agents that are configured to relocate the cell-programming agent to a particular region in the nervous system (e.g., striatum) or a particular type of cells in the nervous system of the subject.

In some embodiments, the cell-programming agent used in the methods provided herein comprises a virus that expresses a Cas effector and an anti-PTB or anti-nPTB gRNA, and the methods comprise injection of the virus in a desired brain region stereotaxically. In some embodiments, the virus comprises adenovirus, lentivirus, adeno-associated virus (AAV), poliovirus, herpes simplex virus (HSV), or murine Maloney-based virus. The AAV that can be used in the methods provided herein can be any appropriate serotype of AAV, such as, but not limited to, AAV2, AAV5, AAV6, AAV7, AAV8, and AAV9. In some embodiments, the methods comprise delivering an AAV2- or AAV9-based viral vector that expresses an agent that suppresses expression or activity of PTB and/or nPTB in a non neuronal cell in a region in nervous system, e.g., brain (e.g., striatum) or spinal cord.

In some embodiments, as described above, the methods provided herein comprise reprogramming a variety of non-neuronal cells to mature neurons. In some embodiments, the methods provided herein comprise administering to a region in the nervous system, e.g., brain (e.g., striatum) or spinal cord, of a subject a composition comprising a cell-programming agent that suppresses the expression or activity of PTB and/or nPTB in a variety of non-neuronal cells, such as, but not limited to, glial cells, e.g., astrocyte, oligodendrocyte, NG2 cell, satellite cell, or ependymal cell in the nervous system, and allowing the non-neuronal cell to reprogram into the functional neuron. In some embodiments, the methods provided herein comprise reprogramming astrocyte in a region in the nervous system, e.g., brain (e.g., striatum) or spinal cord, of a subject into a functional neuron.

As discussed above, the methods provided herein can comprise reprogramming a non-neuronal cell in a specific brain region (e.g., striatum) into a functional neuron. Exemplary brain regions that can be used in the methods provided herein can be in any of hindbrain, midbrain, or forebrain. In some embodiments, the methods provided herein comprise administering to a midbrain, striatum, or cortex of a subject a composition comprising a cell-programming agent that suppresses the expression or activity of PTB in a non-neuronal cell in mature retina or in the striatum, and allowing the non-neuronal cell to reprogram into the functional neuron. In some embodiments, the methods provided herein comprise administering to mature retina or in the striatum of a subject a composition comprising a cell-programming agent that suppresses the expression or activity of PTB/nPTB in a non-neuronal cell in the mature retina or in the striatum, and allowing the non-neuronal cell to reprogram into the functional neuron.

In some embodiments, the methods provided herein comprise reprogramming a non-neuronal cell into a functional neuron in a brain region, such as, but not limited to, medulla oblongata, medullary pyramids, olivary body, inferior olivary nucleus, rostral ventrolateral medulla, caudal ventrolateral medulla, solitary nucleus, respiratory center-respiratory groups, dorsal respiratory group, ventral respiratory group or apneustic centre, pre-bdtzinger complex, botzinger complex, retrotrapezoid nucleus, nucleus retrofacialis, nucleus retroambiguus, nucleus para-ambiguus, paramedian reticular nucleus, gigantocellular reticular nucleus, parafacial zone, cuneate nucleus, gracile nucleus, perihypoglossal nuclei, intercalated nucleus, prepositus nucleus, sublingual nucleus, area postrema, medullary cranial nerve nuclei, inferior salivatory nucleus, nucleus ambiguous, dorsal nucleus of vagus nerve, hypoglossal nucleus, metencephalon, pons, pontine nuclei, pontine cranial nerve nuclei, chief or pontine nucleus of the trigeminal nerve sensory nucleus, motor nucleus for the trigeminal nerve (v), abducens nucleus (vi), facial nerve nucleus (vii), vestibulocochlear nuclei (vestibular nuclei and cochlear nuclei) (viii), superior salivatory nucleus, pontine tegmentum, pontine micturition center (barrington's nucleus), locus coeruleus, pedunculopontine nucleus, laterodorsal tegmental nucleus, tegmental pontine reticular nucleus, parabrachial area, medial parabrachial nucleus, lateral parabrachial nucleus, subparabrachial nucleus (kdlliker-fuse nucleus), pontine respiratory group, superior olivary complex, medial superior olive, lateral superior olive, medial nucleus of the trapezoid body, paramedian pontine reticular formation, parvocellular reticular nucleus, caudal pontine reticular nucleus, cerebellar peduncles, superior cerebellar peduncle, middle cerebellar peduncle, inferior cerebellar peduncle, fourth ventricle, cerebellum, cerebellar vermis, cerebellar hemispheres, anterior lobe, posterior lobe, flocculonodular lobe, cerebellar nuclei, fastigial nucleus, interposed nucleus, globose nucleus, emboliform nucleus, dentate nucleus, midbrain (mesencephalon), tectum, corpora quadrigemina, inferior colliculi, superior colliculi, pretectum, tegmentum, periaqueductal gray, rostral interstitial nucleus of medial longitudinal fasciculus, midbrain reticular formation, dorsal raphe nucleus, red nucleus, ventral tegmental area, parabrachial pigmented nucleus, paranigral nucleus, rostromedial tegmental nucleus, caudal linear nucleus, rostral linear nucleus of the raphe, interfascicular nucleus, substantia nigra, pars compacta, pars reticulata, interpeduncular nucleus, cerebral peduncle, crus cerebri, mesencephalic cranial nerve nuclei, oculomotor nucleus (iii), edinger-westphal nucleus, trochlear nucleus (iv), mesencephalic duct (cerebral aqueduct, aqueduct of sylvius), forebrain (prosencephalon), diencephalon, epithalamus, pineal body, habenular nuclei, stria medullaris, taenia thalami, third ventricle, subcommissural organ, thalamus, anterior nuclear group, anteroventral nucleus (a.k.a. ventral anterior nucleus), anterodorsal nucleus, anteromedial nucleus, medial nuclear group, medial dorsal nucleus, midline nuclear group, paratenial nucleus, reuniens nucleus, rhomboidal nucleus, intralaminar nuclear group, centromedian nucleus, parafascicular nucleus, paracentral nucleus, central lateral nucleus, lateral nuclear group, lateral dorsal nucleus, lateral posterior nucleus, pulvinar, ventral nuclear group, ventral anterior nucleus, ventral lateral nucleus, ventral posterior nucleus, ventral posterior lateral nucleus, ventral posterior medial nucleus, metathalamus, medial geniculate body, lateral geniculate body, thalamic reticular nucleus, hypothalamus (limbic system) (hpa axis), anterior, medial area, parts of preoptic area, medial preoptic nucleus, suprachiasmatic nucleus, paraventricular nucleus, supraoptic nucleus (mainly), anterior hypothalamic nucleus, lateral area, parts of preoptic area, lateral preoptic nucleus, anterior part of lateral nucleus, part of supraoptic nucleus, other nuclei of preoptic area, median preoptic nucleus, periventricular preoptic nucleus, tuberal, medial area, dorsomedial hypothalamic nucleus, ventromedial nucleus, arcuate nucleus, lateral area, tuberal part of lateral nucleus, lateral tuberal nuclei, posterior, medial area, mammillary nuclei, posterior nucleus, lateral area, posterior part of lateral nucleus, optic chiasm, subfornical organ, periventricular nucleus, pituitary stalk, tuber cinereum, tuberal nucleus, tuberomammillary nucleus, tuberal region, mammillary bodies, mammillary nucleus, subthalamus, subthalamic nucleus, zona incerta, pituitary gland, neurohypophysis, pars intermedia (intermediate lobe), adenohypophysis, frontal lobe, parietal lobe, occipital lobe, temporal lobe, cerebellum, brainstem, centrum semiovale, corona radiata, internal capsule, external capsule, extreme capsule, subcortical, hippocampus, dentate gyrus, cornu ammonis (CA fields), cornu ammonis area 1 (CA1), cornu ammonis area 2 (CA2), cornu ammonis area 3 (CA3), cornu ammonis area 4 (CA4), amygdala, central nucleus of amygdala, medial nucleus of amygdala, cortical and basomedial nuclei of amygdala, lateral and basolateral nuclei of amygdala, extended amygdala, stria terminalis, bed nucleus of the stria terminalis, claustrum, basal ganglia, striatum, dorsal striatum, putamen, caudate nucleus, ventral striatum, nucleus accumbens, olfactory tubercle, globus pallidus, ventral pallidum, subthalamic nucleus, basal forebrain, anterior perforated substance, substantia innominata, nucleus basalis, diagonal band of broca, septal nuclei, medial septal nuclei, lamina terminalis, vascular organ of lamina terminalis, rhinencephalon (paleopallium), olfactory bulb, olfactory tract, anterior olfactory nucleus, piriform cortex, anterior commissure, uncus, periamygdaloid cortex, cerebral cortex, frontal lobe, cortex, primary motor cortex (precentral gyms, Ml), supplementary motor cortex, premotor cortex, prefrontal cortex, orbitofrontal cortex, dorsolateral prefrontal cortex, gyri, superior frontal gyms, middle frontal gyms, inferior frontal gyms, Brodmann areas 4, 6, 8, 9, 10, 11, 12, 24, 25, 32, 33, 44, 45, 46, and 47, parietal lobe, cortex, primary somatosensory cortex (SI), secondary somatosensory cortex (S2), posterior parietal cortex, gyri, postcentral gyms (primary somesthetic area), precuneus, Brodmann areas 1, 2, 3, 5, 7, 23, 26, 29, 31, 39, and 40, occipital lobe, cortex, primary visual cortex (VI), v2, v3, v4, v5/mt, gyri, lateral occipital gyms, cuneus, Brodmann areas 17 (VI, primary visual cortex); 18, and 19, temporal lobe, cortex, primary auditory cortex (A1), secondary auditory cortex (A2), inferior temporal cortex, posterior inferior temporal cortex, gyri, superior temporal gyms, middle temporal gyms, inferior temporal gyms, entorhinal cortex, perirhinal cortex, parahippocampal gyms, fusiform gyms, Brodmann areas 20, 21, 22, 27, 34, 35, 36, 37, 38, 41, and 42, medial superior temporal area (MST), insular cortex, cingulate cortex, anterior cingulate, posterior cingulate, retrosplenial cortex, indusium griseum, subgenual area 25, and Brodmann areas 23, 24; 26, 29, 30 (retrosplenial areas); 31, and 32.

In one aspect, the invention provides a method of generating a dopaminergic neuron in vivo. An exemplary method comprises administering to the striatum in the brain of a subject a composition comprising a cell-programming agent (e.g., a Cas effector protein and a coding sequence for gRNA against PTB and/or nPTB) that suppresses expression or activity of PTB and/or nPTB in a non-neuronal cell in the brain (e.g., a glial cell or astrocyte), and allowing the non-neuronal cell to reprogram into the dopaminergic neuron.

In another aspect, the invention provides a method of generating a RGC neuron in vivo. An exemplary method comprises administering to the mature retina of a subject a composition comprising a cell-programming agent (e.g., a Cas effector protein and a coding sequence for gRNA against PTB and/or nPTB) that suppresses expression or activity of PTB and/or nPTB in a non-neuronal cell in the mature retina (e.g., a glial cell or MG cell), and allowing the non-neuronal cell to reprogram into the RGC neuron.

In some embodiments, the methods provided herein comprise administering to a region in the nervous system, e.g., brain or spinal cord, of a subject a composition comprising a cell programming agent (e.g., a Cas effector protein and a coding sequence for gRNA against PTB and/or nPTB) that suppresses the expression or activity of PTB and/or nPTB in a non-neuronal cell in the region, and allowing the non-neuronal cell to reprogram into a functional neuron of a subtype that is predominant in the region.

Without being bound to a particular theory, the methods provided herein can take advantage of local induction signals in a region, e.g., a specific brain region, when reprogramming a non-neuronal cell into a functional neuron in vivo. For example, local signals in the striatum may induce the conversion of non-neuronal cells with PTB/nPTB suppressed into dopamine neurons. Local neurons, non-neuronal cells, e.g., astrocytes, microglia, or both, or other local constituents of the striatum can contribute to the subtype specification of the neuron that is generated from the non-neuronal cell under the induction of the cell-programming agent.

In some embodiments, the methods provided herein comprise administering to a brain region (e.g., striatum) of a subject a composition comprising a cell-programming agent (e.g., a Cas effector protein and a coding sequence for gRNA against PTB and/or nPTB) that suppresses the expression or activity of PTB/nPTB in a plurality of non-neuronal cell in the brain region, and the methods further comprise reprogramming at least about 5%, at least about 10%, at least about 20%, at least about 25%, at least about 30%, at least about 35%, at least about 38%, at least about 40%, at least about 42%, at least about 44%, at least about 46%, at least about 48%, at least about 50%, at least about 52%, at least about 54%, at least about 56%, at least about 58%, at least about 60%, at least about 62%, at least about 64%, at least about 66%, at least about 68%, at least about 70%, at least about 72%, at least about 74%, at least about 76%, at least about 78%, at least about 80%, at least about 82%, at least about 84%, at least about 86%, at least about 88%, at least about 90%, at least about 92%, at least about 94%, at least about 96%, at least about 98%, or at least about 99% of the non-neuronal cells to dopaminergic neurons.

In some embodiments, the methods provided herein comprise administering to the mature retina or a brain region (e.g., striatum) of a subject a composition comprising a cell-programming agent (e.g., a Cas effector protein and a coding sequence for gRNA against PTB and/or nPTB) that suppresses the expression or activity of PTB/nPTB in a plurality of non-neuronal cell in the brain region, and at least about 5%, at least about 10%, at least about 20%, at least about 25%, at least about 30%, at least about 35%, at least about 38%, at least about 40%, at least about 42%, at least about 44%, at least about 46%, at least about 48%, at least about 50%, at least about 52%, at least about 54%, at least about 56%, at least about 58%, at least about 60%, at least about 62%, at least about 64%, at least about 66%, at least about 68%, at least about 70%, at least about 72%, at least about 74%, at least about 76%, at least about 78%, at least about 80%, at least about 82%, at least about 84%, at least about 86%, at least about 88%, at least about 90%, at least about 92%, at least about 94%, at least about 96%, at least about 98%, or at least about 99% of the functional neurons generated by the methods are RGC or dopaminergic, respectively.

In some embodiments, the dopaminergic neuron generated in the methods provided herein expresses one or more markers of dopaminergic neurons, including, but not limited to, dopamine, tyrosine hydroxylase (TH), dopamine transporter (DAT), vesicular monoamine transporter 2 (VMAT2), engrailed homeobox 1 (En1), Nuclear receptor related-1 (Nurr1), G-protein-regulated inward-rectifier potassium channel 2 (Girk2), forkhead box A2 (FoxA2), orthodenticle homeobox 2 (OTX2) and/or LEVI homeobox transcription factor 1 alpha (Lmx1a).

In some embodiments, the dopamine neuron generated in the methods provided herein exhibit Ih current, which can be mediated by Hyperpolarization-activated cyclic nucleotide-gated (HCN) channels. Ih current can be characterized as a slowly activating, inward current, which can be activated by hyperpolarizing steps. For instance, under voltage clamp and the holding potential Vh is −40 mV, an inward slowly activating current can be triggered in a dopamine neuron, with a reversal potential close to −30 mV. The activation curve of Ih current characteristic of a dopamine neuron generated in the methods provided herein can range from −50 to −120 mV with a mid-activation point of −84-1 mV.

In some embodiments, the dopaminergic neurons generated in the methods provided herein have gene expression profile similar to a native dopaminergic neuron.

In some embodiments, the dopaminergic neurons generated in the methods provided herein release dopamine as a neurotransmitter.

A dopaminergic neuron generated in the methods provided herein can be of any subtype of dopaminergic neuron, including, but not limited to, A9 (e.g., immunopositive for Girk2), A10 (e.g., immunopositive for calbindin-D28 k), A11, A12, A13, A16, Aaq, and telencephalic dopamine neurons.

According to some embodiments of the present disclosure, the methods provided herein comprise reprogramming a non-neuronal cell in a region in the nervous system, e.g., mature retina or a region of the brain or spinal cord (e.g., striatum), of a subject to a functional neuron. In some embodiments, the functional neuron as discussed here is integrated into the neural network in the nervous system. As described herein, the reprogrammed functional neuron can form synaptic connections with local neurons, e.g., neurons that are adjacent to the reprogrammed functional neurons. For example, synaptic connections between the reprogrammed neuron and neighboring primary neuron (e.g., glutamatergic neurons), GABAergic interneurons, or other neighboring neurons (e.g., dopaminergic neuron, adrenergic neurons, or cholinergic neurons) can form as the reprogrammed neuron matures in vivo. Among these synaptic connections with local neurons, the reprogrammed functional neuron can be a presynaptic neuron, a postsynaptic neuron, or both.

In some embodiments, the reprogrammed functional neuron sends axonal projections to remote brain regions.

In some embodiments, a reprogrammed functional neuron can integrate itself into one or more existing neural pathways in the brain or spinal cord, for instance, but not limited to, superior longitudinal fasciculus, arcuate fasciculus, uncinate fasciculus, perforant pathway, thalamocortical radiations, corpus callosum, anterior commissure, amygdalofugal pathway, interthalamic adhesion, posterior commissure, habenular commissure, fornix, mammillotegmental fasciculus, incertohypothalamic pathway, cerebral peduncle, medial forebrain bundle, medial longitudinal fasciculus, myoclonic triangle, mesocortical pathway, mesolimbic pathway, nigrostriatal pathway, tuberoinfundibular pathway, extrapyramidal system, pyramidal tract, corticospinal tract or cerebrospinal fibers, lateral corticospinal tract, anterior corticospinal tract, corticopontine fibers, frontopontine fibers, temporopontine fibers, corticobulbar tract, corticomesencephalic tract, tectospinal tract, interstitiospinal tract, rubrospinal tract, rubro-olivary tract, olivocerebellar tract, olivospinal tract, vestibulospinal tract, lateral vestibulospinal tract, medial vestibulospinal tract, reticulospinal tract, lateral raphespinal tract, posterior column-medial lemniscus pathway, gracile fasciculus, cuneate fasciculus, medial lemniscus, spinothalamic tract, lateral spinothalamic tract, anterior spinothalamic tract, spinomesencephalic tract, spinocerebellar tract, spino-olivary tract, and spinoreticular tract. Without being bound to a certain theory, local cellular environment can be correlated with the projections of a functional neuron generated according to some embodiments of the present disclosure. For instance, a functional neuron generated in striatum according to some embodiments of the methods provided herein can be affected by other cells in the local environment of striatum.

### 10. Treatable Conditions/Diseases

In an aspect, the present disclosure provides a method of treating a neurological condition associated with degeneration of functional neurons in a region in the nervous system. An exemplary comprises administering to the region of the nervous system, e.g., mature retina or a region of the brain or spinal (e.g., striatum), of a subject in need thereof a composition comprising a cell-programming agent that suppresses the expression or activity of PTB/nPTB in a non-neuronal cell in the region, and allowing the non-neuronal cell to reprogram into a functional neuron (e.g., RGC or dopaminergic neuron), thereby replenishing the degenerated functional neurons in the region.

According to some embodiments of the present disclosure, methods provided herein comprise treating neurological conditions, including, but not limited to, Parkinson's disease, Alzheimer's disease, Huntington's disease, Schizophrenia, depression, and drug addiction. Applicable neurological conditions can also include disorders associated with neuronal loss in spinal cord, such as, but not limited to, Amyotrophic lateral sclerosis (ALS) and motor neuron disease. The methods provided herein can also find use in treating or ameliorating one or more symptoms of neurodegenerative diseases including, but not limited to, autosomal dominant cerebellar ataxia, autosomal recessive spastic ataxia of Charlevoix-Saguenay, Corticobasal degeneration, Corticobasal syndrome, Creutzfeldt-Jakob disease, fragile X-associated tremor/ataxia syndrome, frontotemporal dementia and parkinsonism linked to chromosome 17, Kufor-Rakeb syndrome, Lyme disease, Machado-Joseph disease, Niemann-Pick disease, pontocerebellar hypoplasia, Refsum disease, pyruvate dehydrogenase complex deficiency, Sandhoff disease, Shy-Drager syndrome, Tay-Sachs disease, and Wobbly hedgehog syndrome.

As provided herein, “neurodegeneration” or its grammatical equivalents, can refer to the progressive loss of structure, function, or both of neurons, including death of neuron. Neurodegeneration can be due to any type of mechanisms. A neurological condition the methods provided herein are applicable to can be of any etiology. A neurological condition can be inherited or sporadic, can be due to genetic mutations, protein misfolding, oxidative stress, or environment exposures (e.g., toxins or drugs of abuse).

In some embodiments, the methods provided herein treat a neurological condition associated with degeneration of dopaminergic neurons in a brain region. In some embodiments, the methods provided herein treat a neurological condition associated with degeneration of RGC neurons in the mature retina. In other embodiments, the methods provided herein treat a neurological condition associated with degeneration of any type of neurons, such as, but not limited to, glutamatergic neurons, GABAergic neurons, cholinergic neurons, adrenergic neurons, dopaminergic neurons, or any other appropriate type neurons that release neurotransmitter aspartate, D-serine, glycine, nitric oxide (NO), carbon monoxide (CO), hydrogen sulfide (H2S), norepinephrine (also known as noradrenaline), histamine, serotonin, phenethylamine, N-methylphenethylamine, tyramine, 3-iodothyronamine, octopamine, tryptamine, somatostatin, substance P, opioid peptides, adenosine triphosphate (ATP), adenosine, or anandamide. The methods provided herein can find use in treating a neurological condition associated with neuronal degeneration in any region, such as, but limited to, midbrain regions (e.g., substantial nigra or ventral tegmental area), forebrain regions, hindbrain regions, or spinal cord. The methods provided herein can comprise reprogramming non-neuronal cells to functional neurons in any appropriate region (s) in the nervous system in order to treat a neurological condition associated with neuronal degeneration.

Methods provided herein can find use in treating or ameliorating one or more symptoms associate with Parkinson's disease. Parkinson's disease is a neuro-degenerative disease with early prominent functional impairment or death of dopaminergic neurons in the substantia nigra pars compacta (SNpc). The resultant dopamine deficiency within the basal ganglia can lead to a movement disorder characterized by classical parkinsonian motor symptoms. Parkinson's disease can also be associated with numerous non-motor symptoms. One standard for diagnosis of Parkinson's disease can be the presence of SNpc degeneration and Lewy pathology at post-mortem pathological examination. Lewy pathology can include abnormal aggregates of a-synuclein protein, called Lewy bodies and Lewy neurites. Patients with Parkinson's disease can exhibit a number of symptoms, including motor symptoms and non motor symptoms. Methods provided herein can treat or ameliorate one or more of these motor or non-motor symptoms associated with Parkinson's disease. Motor symptoms of Parkinson's disease (Parkinsonism symptoms) can include bradykinesia (slowness), stiffness, impaired balance, shuffling gait, and postural instability. Motor features in patients with Parkinson's disease can be heterogeneous, which has prompted attempts to classify subtypes of the disease, for instance, tremor-dominant Parkinson's disease (with a relative absence of other motor symptoms), non-tremor-dominant Parkinson's disease (which can include phenotypes described as akinetic-rigid syndrome and postural instability gait disorder), and an additional subgroup with a mixed or indeterminate phenotype with several motor symptoms of comparable severity. Non motor symptoms of Parkinson's disease can include olfactory dysfunction, cognitive impairment, psychiatric symptoms (e.g., depression), sleep disorders, autonomic dysfunction, pain, and fatigue. These symptoms can be common in early Parkinson's disease. Non-motor features can also be frequently present in Parkinson's disease before the onset of the classical motor symptoms. This premotor or prodromal phase of the disease can be characterized by impaired olfaction, constipation, depression, excessive daytime sleepiness, and rapid eye movement sleep behavior disorder.

In some embodiments, methods provided herein mitigate or slow the progression of Parkinson's disease. Progression of Parkinson's disease can be characterized by worsening of motor features. As the disease advances, there can be an emergence of complications related to long-term symptomatic treatment, including motor and non-motor fluctuations, dyskinesia, and psychosis.

One pathological feature of Parkinson's disease can be loss of dopaminergic neurons within the substantial nigra, e.g., substantial nigra pars compacta (SNpc). According to some embodiments, methods provided herein replenish dopamine (secreted from converted dopamine neuron in the striatum) diminished due to loss of dopamine neuron in substantial nigra (e.g., SNpc) of a patient. Neuronal loss in Parkinson's disease can also occur in many other brain regions, including the locus ceruleus, nucleus basalis of Meynert, pedunculopontine nucleus, raphe nucleus, dorsal motor nucleus of the vagus, amygdala, and hypothalamus. In some embodiments, methods of treating or ameliorating one or more symptoms of Parkinson's disease in a subject as provided herein include reprogramming non-neuronal cells to functional neurons in brain regions experiencing neuronal loss in a patient with Parkinson's disease.

Methods provided herein can find use in treating Parkinson's disease of different etiology. For example, there can be Parkinson's disease as a result of one or more genetic mutations, such as, but not limited to, mutations in genes SNCA, LRRK2, VPS35, EIF4G1, DNAJC13, CHCHD2, Parkin, PINK1, DJ-1, ATP13A2, C90RF72, FBX07, PLA2G6, POLG1, SCA2, SCA3, SYNJ1, RAB39B, and possibly one or more genes affected in 22q11.2 microdeletion syndrome. Or there can be Parkinson's disease with no known genetic traits.

As provided herein, the one or more symptoms of Parkinson's disease the methods provided herein can ameliorate can include not only the motor symptoms and non-symptoms as described above, but also pathological features at other levels. For example, reduction in dopamine signaling in the brain of a patient with Parkinson's disease can be reversed or mitigated by methods provided herein by replenishing functional dopamine neurons, which can be integrated into the neural circuitry and reconstruct the dopamine neuron projections to appropriate brain regions.

In an aspect, the present disclosure also provides methods of restoring dopamine release in subject with a decreased amount of dopamine biogenesis compared to a normal level. An exemplary method comprises reprogramming a non-neuronal cell in a brain region of the subject (e.g., striatum), and allowing the non-neuronal cell to reprogram into a dopaminergic neuron, thereby restoring at least 50% of the decreased amount of dopamine. In some embodiments, the reprogramming is performed by administering to the brain region of the subject (e.g., striatum) a composition comprising a cell-programming agent that suppresses the expression or activity of PTB/nPTB in a non-neuronal cell (e.g., an astrocyte) in the brain region. In some embodiments, the methods provided herein restore at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 98% of the decreased amount of dopamine. In some embodiments, the methods provided herein restore about 20%, about 25%, about 30%, about 35%, about 40%, about 45%, about 50%, about 55%, about 60%, about 65%, about 70%, about 75%, about 80%, about 85%, about 90%, about 95%, about 98%, or about 100% of the decreased amount of dopamine. In some embodiments, the methods provided herein restore at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 98% of the decreased amount of dopamine. In some embodiments, the methods provided herein restore at least about 50% of the decreased amount of dopamine.

### 11. Pharmaceutical Composition

In one aspect, the present disclosure provides pharmaceutical compositions comprising a cell-programming agent in an amount effective to reprogram a mammalian non-neuronal cell to a mature neuron by suppressing the expression or activity of PTB/nPTB in the non-neuronal cell. An exemplary pharmaceutical composition can further comprise a pharmaceutically acceptable carrier or excipient. As described above, a cell-programming agent as provided herein can be a Cas effector protein and a coding sequence for a gRNA against PTB/nPTB.

A pharmaceutical composition provided herein can include one or more carriers and excipients (including but not limited to buffers, carbohydrates, mannitol, proteins, peptides or amino acids such as glycine, antioxidants, bacteriostats, chelating agents, suspending agents, thickening agents and/or preservatives), water, oils including those of petroleum, animal, vegetable or synthetic origin, such as peanut oil, soybean oil, mineral oil, sesame oil and the like, saline solutions, aqueous dextrose and glycerol solutions, flavoring agents, coloring agents, detackifiers and other acceptable additives, or binders, other pharmaceutically acceptable auxiliary substances as required to approximate physiological conditions, such as pH buffering agents, tonicity adjusting agents, emulsifying agents, wetting agents and the like. Examples of excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. In another instance, the composition is substantially free of preservatives. In other embodiments, the composition contains at least one preservative. General methodology on pharmaceutical dosage forms can be found in Ansel et ah, Pharmaceutical Dosage Forms and Drug Delivery Systems (Lippencott Williams & Wilkins, Baltimore Md. (1999)). It will be recognized that, while any suitable carrier known to those of ordinary skill in the art can be employed to administer the pharmaceutical compositions described herein, the type of carrier can vary depending on the mode of administration. Suitable formulations and additional carriers are described in Remington “The Science and Practice of Pharmacy” (20th Ed., Lippincott Williams & Wilkins, Baltimore Md.), the teachings of which are incorporated by reference in their entirety herein.

An exemplary pharmaceutical composition can be formulated for injection, inhalation, parenteral administration, intravenous administration, subcutaneous administration, intramuscular administration, intradermal administration, topical administration, or oral administration.

In certain embodiments, the pharmaceutical composition comprising an AAV vector encoding a Cas effector and a coding sequence for a gRNA against PTB/nPTB can be injected into the mature retina, or the striatum of a subject's brain.

As one of ordinary skills in the art will appreciate, pharmaceutical compositions can comprise any appropriate carrier or excipient, depending on the type of cell programming agent and the administration route the composition is designed for. For example, a composition comprising a cell programming agent as provided herein can be formulated for parenteral administration and can be presented in unit dose form in ampoules, pre-filled syringes, small volume infusion or in multi dose containers with an added preservative. The composition can take such forms as suspensions, solutions, or emulsions in oily or aqueous vehicles, for example solutions in aqueous polyethylene glycol. For example, for injectable formulations, a vehicle can be chosen from those known in the art to be suitable, including aqueous solutions or oil suspensions, or emulsions, with sesame oil, corn oil, cottonseed oil, or peanut oil, as well as elixirs, mannitol, dextrose, or a sterile aqueous solution, and similar pharmaceutical vehicles. The formulation can also comprise polymer compositions which are biocompatible, biodegradable, such as poly (lactic-co-glycolic) acid. These materials can be made into micro or nanospheres, loaded with drug and further coated or derivatized to provide superior sustained release performance.

Vehicles suitable for periocular or intraocular injection include, for example, suspensions of active agent in injection grade water, liposomes, and vehicles suitable for lipophilic substances and those known in the art. A composition as provided herein can further comprise additional agent besides a cell-programming agent and a pharmaceutically acceptable carrier or excipient. For example, additional agent can be provided for promoting neuronal survival purpose. Alternatively or additionally, additional agent can be provided for monitoring pharmacodynamics purpose. In some embodiments, a composition comprises additional agent as a penetration enhancer or for sustained release or controlled release of the active ingredient, e.g., cell-programming agent.

A composition provided herein can be administered to a subject in a dosage volume of about 0.0005, 0.001, 0.002, 0.005, 0.01, 0.02, 0.05, 0.1, 0.15, 0.2, 0.25, 0.3, 0.35, 0.4, 0.45, 0.5, 0.55, 0.6, 0.7, 0.8, 0.9, 1.0 mL, or more. The composition can be administered as a 1, 2, 3, 4, 5, 6, 7, 8, 9, 10, or more dose-course regimen. Sometimes, the composition can be administered as a 2, 3, or 4 dose-course regimen. Sometimes the composition can be administered as a 1 dose-course regimen.

The administration of the first dose (e.g., an AAV vector encoding a Cas effector and a gRNA against PTB) and second dose (e.g., an AAV vector encoding a Cas effector and a gRNA against nPTB) of the 2 dose-course regimen can be separated by about 0 day, 1 day, 2 days, 5 days, 7 days, 14 days, 21 days, 30 days, 2 months, 4 months, 6 months, 9 months, 1 year, 1.5 years, 2 years, 3 years, 4 years, 5 years, 10 years, 20 years, or more. A composition described herein can be administered to a subject once a day, once a week, once two weeks, once a month, a year, twice a year, three times a year, every 1, 2, 3, 4, 5, 6, 7, 8, 9, 10, or more years.

Sometimes, the composition can be administered to a subject every 2, 3, 4, 5, 6, 7, or more years. Sometimes, the composition can be administered to a subject once.

### 12. Further Aspects

Some embodiments of the disclosure provide methods and compositions for cell or tissue transplantation. An exemplary method can comprise reprogramming a non-neuronal cell to a neuron in vitro, and transplanting the reprogrammed neuron into a brain region in a subject. In some embodiments, in vitro reprogramming can be performed according to the methods provided herein. An exemplary composition can comprise a neuron reprogrammed according to any embodiment of the methods provided herein.

In other embodiments, a method provided herein comprises reprogramming a non-neuronal cell to a neuron in vivo, and explanting the reprogrammed neuron. In some embodiments, the explant comprises a brain tissue comprising the reprogramed neuron. In some embodiments, the explant is transplanted into a brain region of a subject. As provided herein, the transplantation of neurons reprogrammed according to the methods provided herein can be used to replenish degenerated neurons in a subject suffering a condition associated with neuronal loss.

Some other aspects of the present disclosure relate to an animal that comprise neurons reprogrammed according to any embodiment of the methods provided herein.

As provided herein, an animal can be any mammal. An animal can be a human. An animal can be a non-human primate, such as, but not limited, rhesus macaques, crab-eating macaques, stump-tailed macaques, pig-tailed macaques, squirrel monkeys, owl monkeys, baboons, chimpanzees, marmosets and spider monkeys. An animal can be a research animal, a genetically modified animal, or any other appropriate type of animal. For example, a mouse or rat can be provided that comprises one or more neurons reprogrammed according to an embodiment of the present disclosure.

Also provided herein is a brain tissue (e.g., explant) of an animal that comprises one or more neurons reprogrammed according to any embodiment of the present disclosure. Such brain tissue can be live. In some embodiments, a brain tissue can be fixed by any appropriate fixative. A brain tissue can be used for transplantation, medical research, basic research, or any type of purposes.

The disclosure demonstrates that the method is applicable to disease models of neurodegeneration. For example, the disclosure shows that astrocyte-to-neuron conversion strategy can work in a chemical-induced Parkinson's disease model. The methods and compositions can convert astrocytes to neurons including dopaminergic, glutamatergic and GABAergic neurons, these neurons are able to form synapses in the brain, and remarkably, the converted neurons can efficiently reconstruct the lesioned nigrostriatal pathway to correct measurable Parkinson's phenotypes. The effectiveness of this method was demonstrated both in astrocytes in culture (human and mouse) as well as in vivo in a mouse Parkinson's disease model. Therefore, this strategy has the potential to cure Parkinson's disease, which can also be applied to a wide range of neurodegenerative diseases (e.g., other neurological diseases associated with neuronal dysfunction).

In some embodiments, the approach of the disclosure exploits the genetic foundation of a neuronal maturation program already present, but latent, in both mammalian astrocytes that progressively produce mature neurons once they are reprogrammed by PTB suppression. These findings provide a clinically feasible approach to generate neurons from local astrocytes in mammalian brain using a single dose of a vector comprising coding sequence for a Cas effector and a gRNA against PTB/nPTB. The phenotypes of PTB/nPTB knockdown-induced neurons can be a function of the context in which they are produced and/or the astrocytes from which they are derived.

The disclosure demonstrates the potent conversion of astrocytes to neurons (e.g., dopamine neurons in the striatum). More particularly, the disclosure shows that in a chemically-induced mouse Parkinson's disease (PD) model, the strategy efficiently can correct a PD phenotype, thus satisfying all five factors for in vivo reprogramming.

The data provided herein show that PTB reduction in the mammalian brain can convert astrocytes to neurons (e.g., dopaminergic neurons) and the reversal of behavioral deficits (e.g., in a chemically-induced PD model).

A “therapeutically effective amount” of a composition of the disclosure will vary according to factors such as the disease state, age, sex, and weight of the individual, and the ability of the composition to elicit a desired response in the individual. A therapeutically effective amount can also be one in which any toxic or detrimental effects of the composition are outweighed by the therapeutically beneficial effects. Without wishing to be bound by a particular theory, it is contemplated that, in some cases, a therapeutically effective amount of cell-programming agent as provided herein can be an amount of cell-programming agent that converts a certain proportion of astrocytes in a brain region that experiences neuronal loss, conversion of such proportion of astrocytes to functional neurons in the brain region is sufficient to ameliorate or treating the disease or condition associated with the neuronal loss in the brain region, and meanwhile, such proportion of astrocytes does not exceed a threshold level that can lead to aversive effects that can overweigh the beneficial effects brought by the neuronal conversion, for instance, due to excessive reduction in the number of astrocytes in the brain region as a direct consequence of the neuronal conversion.

The following examples are intended to illustrate but not limit the disclosure. While they are typical of those that might be used, other procedures known to those skilled in the art may alternatively be used.

Guide RNA sequence
gRNA #1:
(SEQ ID NO: 1)
5′-tttgtaccgactgctatgtctgggacgat-3′;
gRNA #2:
(SEQ ID NO: 2)
5′-ggctggctgtctccagagggcaggtcaggt-3′;
gRNA #3:
(SEQ ID NO: 3)
5′-gtatagtagttaaccatagtgttggcagcc-3′;
gRNA #4:
(SEQ ID NO: 4)
5 ′-gctgtcggtcttgagctctttgtggttgga-3′;
gRNA #5:
(SEQ ID NO: 5)
5′-tgtagatgggctgtccacgaagcactggcg-3′;
gRNA #6:
(SEQ ID NO: 6)
5′-gcttggagaagtcgatgcgcagcgtgcagc-3′.

### EXAMPLES

### Example 1 Ptbp1 Knockdown Converts MG to RGCs in Mature Retinas

Degeneration of retinal ganglion cells (RGCs), the sole output neurons of the retina, represents the leading cause of retinal diseases with permanent blindness. Trans-differentiation of Müller glia (MG) into RGCs has been proposed to be a potential therapy for restoring visual function. However, MG lose the neurogenic capacity at about two postnatal weeks in mice, and MG-to-RGC conversion has not been achieved in mature mammalian retinas so far.

This example demonstrates that MG-to-RGC conversion can be achieved in vivo in mature retina to generate functional RGCs, thus at least partially restoring visual function.

This experiment utilized an orthologue of CRISPR-Cas13d (CasRx), which has the smallest size among the previously known Cas effector proteins, and which exhibits high targeting specificity and efficiency, as an ideal tool for in vivo gene therapeutic application. The small size of CasRx permits it to be encoded by a safe and widely used gene therapy vector—AAV vector (which has a limited packaging capacity of under 5 kb)—together with coding sequence for one or more guide RNA's also required for Cas-mediated mRNA knock down.

Using this approach, this experiment, demonstrated that MG can be efficiently converted into RGCs by injecting AAVs expressing CasRx and two guide RNAs (gRNAs) targeting Ptbp1 mRNA in both intact and damaged mature retinas. The converted RGCs established central projections to dorsal lateral geniculate nucleus (dLGN) and superior colliculus (SC), and partially restored visual functions in a mouse model with drug-induced retinal injury.

An in vitro experiment was first conducted to verify the efficiency of CasRx-mediated knockdown of Ptbp1. Specifically, six potential guide RNAs (gRNAs) were screened for their efficiency in CasRx editing of Ptbp1 in both N2a cells and cultured astrocytes: gRNA #1 targets a region in exon II; gRNA #3, 4, and 5 target regions in exon IV; and gRNA #2 and 6 target regions in exon VII. It was found that co-transfection of a vector containing CasRx gene with two gRNAs 5 and 6 (that target Ptbp1 exon IV and VII, respectively) resulted in 87±0.4 and 76±4% (SEM, n=5 repeats) reduction of Ptbp1 mRNA in N2a cells and cultured astrocytes, respectively (FIGS. 1A and 1B).

Transcriptome analysis showed that Ptbp1 was specifically down-regulated while the transcriptional level of typical neuronal genes remained unchanged, two days after transfection (data not shown).

Having confirmed that the combination of gRNA 5 and 6 provide the optimal knock down of the target gene, the following in vivo experiment was conducted in mice to show that Ptbp1 knockdown in the mature retina could result in conversion of MG into RGCs in vivo.

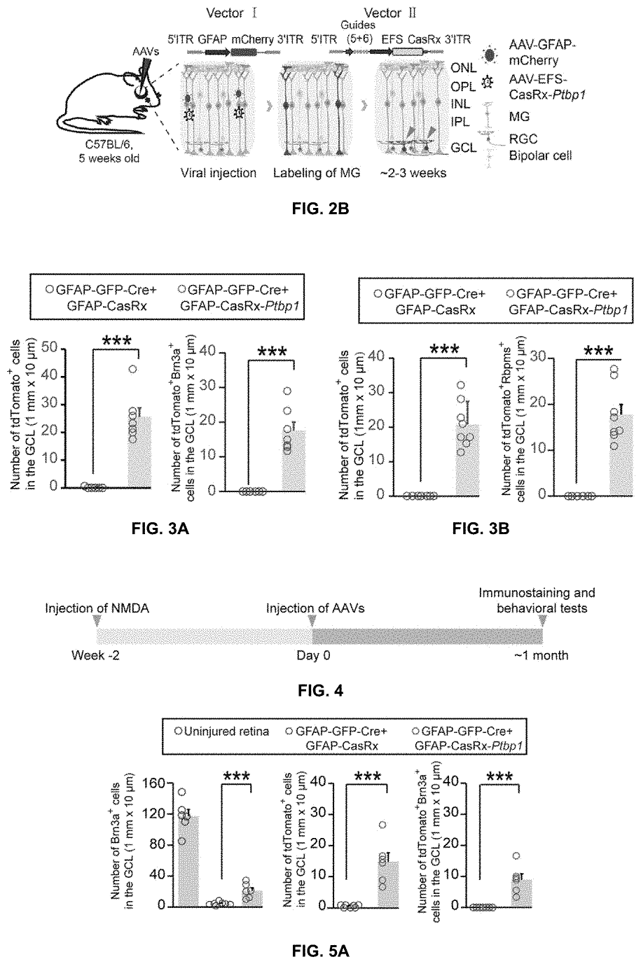

To specifically and permanently label the retinal MG, AAV-GFAP-GFP-Cre vector was injected into the eyes of Ai9 mice (Rosa-CAG-LSL-tdTomato-WPRE) to induce tdTomato expression specifically in MGs (data not shown). Another construct, AAV-GFAP-CasRx-Ptbp1, having gRNAs 5+6 targeting Ptbp1 driven by the GFAP promotor, was constructed to knockdown Ptbp1 specifically in MG. As a negative control, AAV-GFAP-CasRx, which did not contain Ptbp1 gRNAs, was produced (FIG. 2A).

At one-month after subretinal co-injection of AAV-GFAP-CasRx-Ptbp1 and AAV-GFAP-Cre-GFP into the eyes of 5-week Ai9 mice retinas, many tdTomato⁺ cells co-immunostained with RGC markers Brn3a or Rbpms in retinal ganglion cell layer (GCL) (tdTomato⁺ Brn3a⁺, 18±2 cells per 1 mm×10 μm; tdTomato⁺ Rbpms⁺, 18±2 cells per 1 mm×10 μm), but no such cell in the retina injected with control AAV vectors (FIGS. 3A and 3B), suggesting conversion of MG to RGCs in the mature retina.

Notably, converted RGC cells frequently showed low expression of GFAP-driven GFP (data not shown), consistent with the loss of glial identity after MG-to-RGC conversion. Interestingly, a fraction of tdTomato⁺ cells in the GCL expressed Foxp2⁺, Brn3c⁺ or Parvalbumin⁺ (data not shown), markers of F-RGCs, RGCs subtype 3 and PV-RGCs, respectively, suggesting that MG were converted into different subtypes of RGCs.

Successful induction of RGCs was also confirmed by another strategy at 2-3 weeks after co-injecting AAV-GFAP-mCherry and AAV-EFS-CasRx-Ptbp1 (CasRx driven by the ubiquitous promoter EFS) into the retinas of C57BL/6 mice (FIG. 2B).

Together, these results showed that RGCs could be efficiently converted from MG via Ptbp1 knockdown in the mature retina.

It was also found that, besides RGCs, MG could also be converted into amacrine cells by CasRx-mediated knockdown of Ptbp1 (data not shown).

### Example 2 MG-to-RGC Conversion in a NMDA-Induced Retinal Injury Mouse Model

This experiment demonstrates that MG-derived RGCs could replenish RGCs in the NMDA-induced retina injury mouse model.

According to this mouse model, about 4-8 weeks old Ai9 mice were intravitreally injected with N-methyl-D-aspartate (NMDA, 200 mM), which causes a near complete loss of RGCs and the reduction of the thickness of inner plexiform layer (IPL).

Intravitreal injection was performed as previously described. Specifically, the pipettes were prepared using a puller and connected with a 1 ml syringe. Then mice were anaesthetized with 0.35 mL 2% Tribromoethanol, and one drop of 0.5% alcaine was dropped on the eye before intravitreous injection. Around 1.5 μl NMDA solution (200 mM) was injected into the vitreous body using the pipette. After injection, ofloxacin eye ointment was applied on the eye to prevent infection. For subretinal injection, mice were aneathetized and the pupil size was dilated with Tropicamide Phenylephrine Eye drops. Then one drop of sodium hyaluronate was dropped on the cornea to enable better visualization. A penetration was made in the cornea under a Olympus microscope (Olympus, Tokyo, Japan) using a 30 G needle. Next, a Hamilton syringe (32 G needle) was inserted into the eye via corneal perforation. To inject AAVs (high titer: >1×10¹³ vg/ml) into the subretinal space, an inner retina area with low density of blood vessels was penetrated with the needle and ˜1 μL content was injected into the subretinal space with slow speed (taking up to 20 seconds). After injection, the injection needle was removed slowly and a drop of ofloxacin eye ointment was administrated.

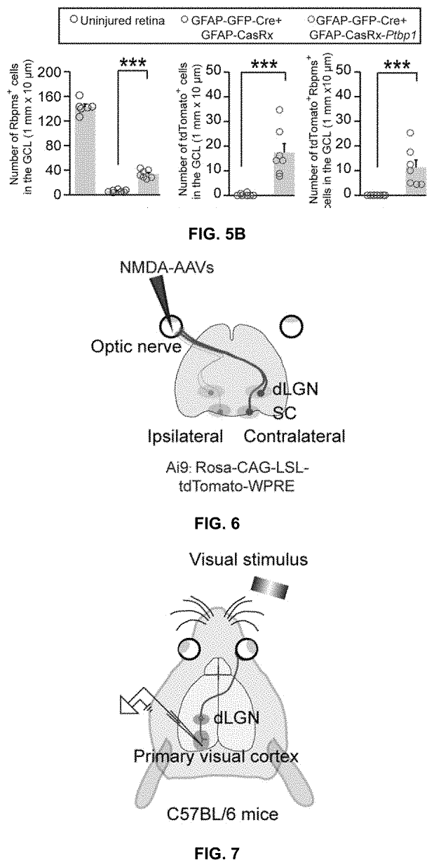

Two to three weeks after NMDA injection, the eyes were either injected with AAV-GFAP-CasRx-Ptbp1 plus AAV-GFAP-GFP-Cre or control AAVs (FIG. 4). One month after AAV injection, the number of Brn3a⁺ or Rbpms⁺ cells (Brn3a⁺, 21±4 cells per 1 mm×10 as compared to 4±1 cells per 1 mm×10 μm in untreated injured retina and 117±8 cells per 1 mm×10 μm in uninjured retina; Rbpms⁺, 34±3 cells per 1 mm×10 as compared to 6±1 cells per 1 mm×10 μm in untreated injured retina and 143±5 cells per 1 mm×10 μm in uninjured retina) in the GCL was significantly elevated in retinas injected with AAV-GFAP-CasRx-Ptbp1, and the majority of these cells were tdTomato⁺ (FIGS. 5A and 5B). Moreover, more than half the tdTomato⁺ cells in GCL expressed Brn3a and Rbpms (FIGS. 5A and 5B).

To determine whether MG-derived RGCs integrate into the retinal circuits and have the capacity of receiving visual information, cell-attached recording from MG-derived RGCs was performed under two-photon microscope to monitor light stimulus-evoked responses (data not shown). It was found that 6 out of 8 cells examined showed action potentials in response to light stimulation (data not shown). Among these cells, five were ON cells and one was OFF cell (data not shown). These results suggested that functional RGCs could be converted from MG via Ptbp1 knockdown in the injured retina.

### Example 3 Central Projections of Converted RGCs Restored Visual Responses

This examples shows that RGC converted from MG cells are functional and can restore visual response.

In the mammalian visual system, RGC projections relay visual information to the dorsal lateral geniculate nucleus (dLGN) and superior colliculus (SC) in the brain (FIG. 6). In the CasRx-treated NMDA-injured retina, a large amount of tdTomato⁺ axons were observed in the treated retina and the optic nerve, but no such axons in control AAV-treated group (data not shown). Remarkably, tdTomato⁺ axons were found in the dLGN and SC, which were much more abundant in the contralateral than the ipsilateral side of the brain (data not shown), consistent with expectation that newly formed axon projections of the converted RGCs correctly send their projections to their central target areas.

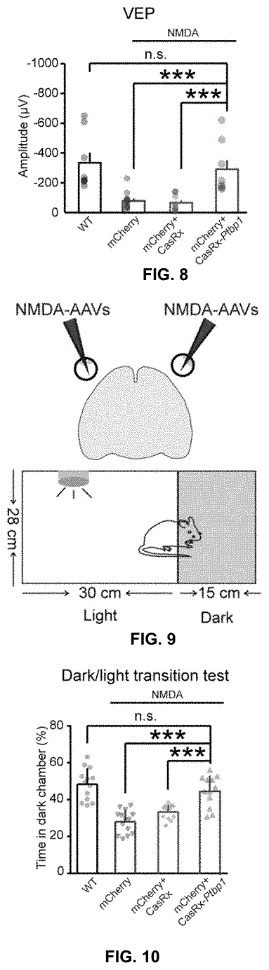

The function of central projections of MG-derived RGCs was further examined by monitoring visual responses evoked by the light stimulus applied to the NMDA-injured retina. Visually evoked potentials (VEPs) were recorded in the primary visual cortex (V1) of anaesthetized mice one month after AAVs injection (FIG. 7). Striking VEPs were evoked by stimulus to the contralateral retina in NMDA-injured mice treated with AAV-GFPA-CasRx-Ptbp1 and AAV-GFAP-mCherry, similar to that found in wild-type un-injured mice, whereas only weak responses were observed in the control NMDA-injured mice injected with control AAV vectors (FIG. 8). This supports the notion that central projection to dLGN had restored at least partially visual information relay to V1, presumably by making synaptic connections with existing functional dLGN neurons in the brain.

Finally, CasRx-mediated conversion of MG to RGCs also restored vision-dependent behavior that was lost by NMDA-induced retinal injury (FIG. 9). Bilateral intravitreal NMDA injection in mice resulted in a reduced duration in the dark compartment in a light/dark preference test, consistent with a loss of vision due to retina injury. By contrast, CasRx-mediated MG-to-RGC conversion in both eyes of bilaterally retina-injured mice resulted in a marked increase in the duration in dark compartment, to a level close to that found for control un-injured mice (FIG. 10), consistent with the restoration of vision-dependent behaviors.

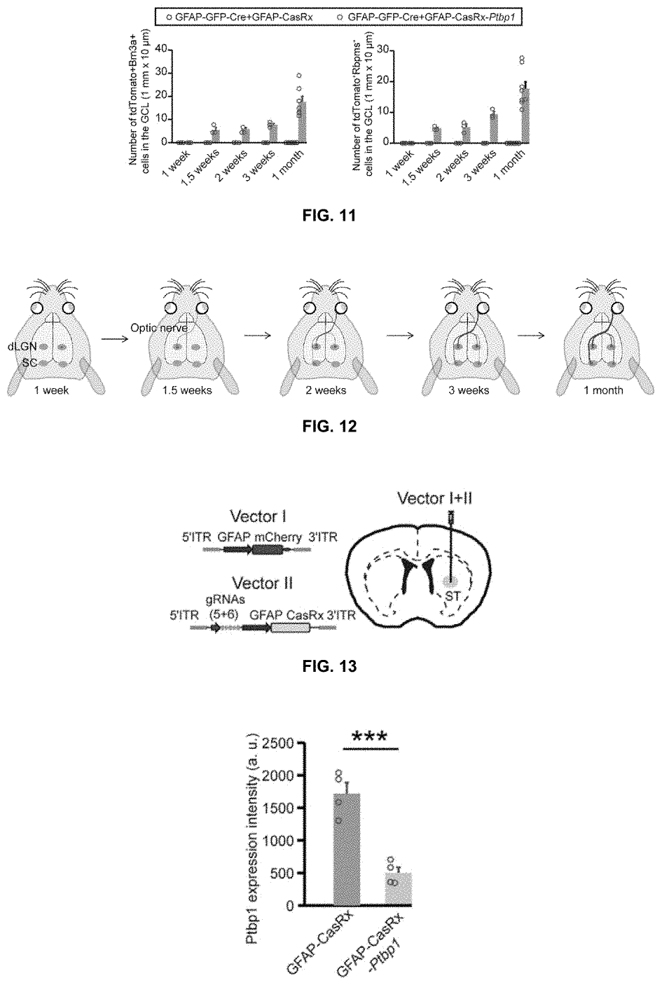

Next, the time-course of appearance of MG-to-RGC conversion and central projections in the intact retinas without NMDA-induced injury were determined, by performing immunostaining at five different time points (1 week, 1.5 weeks, 2 weeks, 3 weeks and 1 month) after AAV injection. tdTomato⁺ Rbpms⁺ and tdTomato⁺ Brn3a⁺ cells in the retina were first seen at 1.5 week after the AAV injection and the number of these cells progressively increased over time (FIG. 11). There was an intermediate stage showing that induced RGCs migrated from INL to GCL at 1.5 week after the AAV injection (data not shown).

The time course of MG-to-RGC conversion was also demonstrated in the retina with NMDA-induced injury (data not shown). For the central projection, progressive increase in the tdTomato⁺ axons in the visual pathway was observed: labeled axons were not found in the optic nerve at 1 week (data not shown) and began to appear in contralateral dLGN at 1.5 week (data not shown) after the AAV injection. Labeled projections were first observed in the contralateral but not ipsilateral SC by 2 weeks (data not shown), and clearly observed in both contralateral and ipsilateral dLGN and SC by 3 weeks, with further increased on both sides at one month (FIG. 12). Similar findings were also observed in the injured retinas injected with NMDA (data not shown).

### Example 4 CasRx-Induced Astrocyte-to-Neuron Conversion in Mouse Striatum

This example demonstrates that Cas-induced glia-to-neuron conversion is not only effective to produce functional neurons in mature retina to at least partially restore lost vision, but also functions similarly in other systems, thus having a more generalized therapeutic application to treat other neurodegenerative diseases.

Specifically, this experiment shows that CasRx-induced Ptbp1 knock-down in the striatum could locally convert other types of cells into dopamine neurons, an approach that can be used for replenishing dopamine in the straitum due to degeneration of dopaminergic neurons in midbrain substantia nigra associated with Parkinson's disease (PD).

Wild-type mice were first injected with AAV-GFAP-CasRx-Ptbp1 (with gRNAs 5+6 for Ptbp1) into the striatum to specifically knockdown Ptbp1, together with AAV-GFAP-mCherry that fluorescently labeled astrocytes (FIG. 13). As a control, AAV-GFAP-CasRx that does not contain Ptbp1 gRNA were injected. Both mCherry and CasRx were largely specifically expressed in astrocytes, and showed a high co-infection efficiency in the striatum, with 99±1% mCherry⁺ cells expressed CasRx (82±2% GFAP⁺ cells expressed mCherry, and 95±1% mCherry⁺ cells expressed GFAP). The absolute number of CasRx-infected cell was 40±8 Flag⁺ cells per 200 μm×200 μm×10 μm (data not shown).

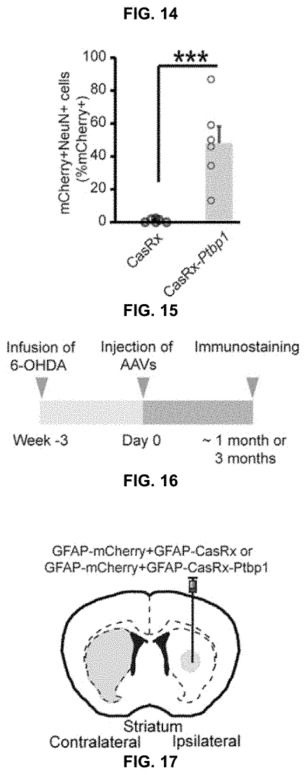

The expression of Ptbp1 was down-regulated in astrocytes one week after co-injecting AAV-GFAP-CasRx-Ptbp1 and AAV-GFAP-mCherry into the striatum (FIG. 14), and a high percentage (48±10%, SEM, n=6 mice) of mCherry⁺ cells expressed mature neuron markers NeuN at one month after AAV injection but not in the control striatum injected with AAV-GFAP-mCherry and AAV-GFAP-CasRx (0.97±0.45%, SEM, n=6 mice) (FIG. 15).

The neuronal type of these converted cells was further examined by immunostaining of cell type-specific markers. Around 50% of converted neurons expressed glutaminase (data not shown), a marker of excitatory glutamatergic neurons, and very few converted neurons expressed an interneuron subtype marker somatostatin (SST), and no cell expressed another interneuron subtype marker parvalbumin (PV) (data not shown). Co-staining of dopamine neuron marker tyrosine hydroxylase (TH) with NeuN showed that a fraction of (7.5±3%, SEM) mCherry⁺ cells expressed TH, but contained a low level of NeuN (data not shown), similar to that found previously in rodent midbrain TH⁺ dopamine neurons.

Furthermore, expression of AAV-GFAP-mCherry in converted neurons persisted for at least one month after infection (data not shown).

### Example 5 Conversion of Striatal Astrocytes into Dopamine Neurons in PD Model Mice

This example shows that the dopamine neurons converted from striatal astrocytes are functional in the mouse model of PD.

This mouse model was generated by unilateral infusion of 6-hydroxydopamine (6-OHDA) into the right medial forebrain bundle. In brief, adult C57BL/6 mice (aged ˜10 weeks) received i.p. injection of 25 mg/kg of Desipramine hydrochloride half-hour before anesthesia. After anesthesia, mice were injected with 3 μg 6-OHDA or saline into right medial forebrain bundle according to the following coordinates: anteroposterior (A/P)=−1.2 mm, mediolateral (M/L)=−1.1 mm, dorsoventral (DN)=−5 mm. All mice were Formatted: Font: (Default) Arial, 12 pt delivered 1 ml of 4% glucose-saline solution subcutaneously 1 hour after surgery. Mice were typically allowed to recover for 3 weeks feeding with soaked food pellets.

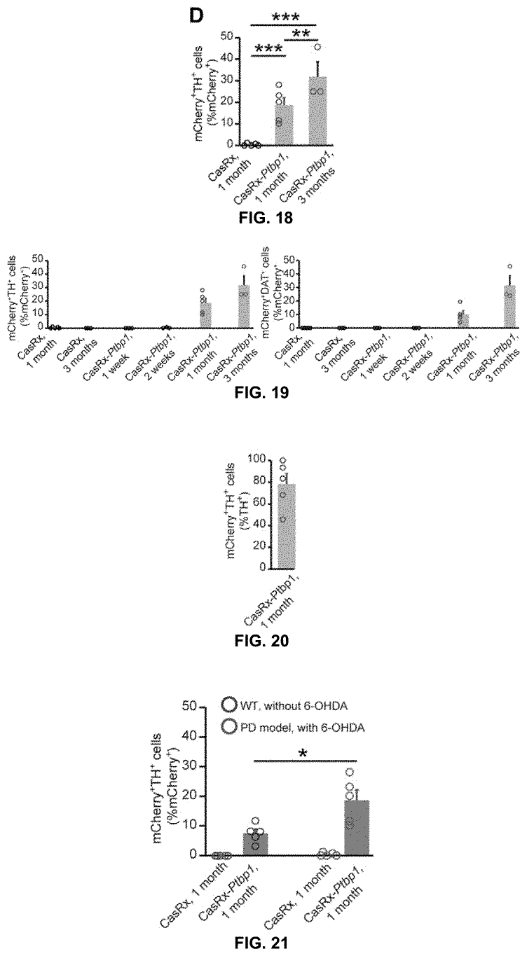

This infusion induces the loss of dopamine neurons in the ipsilateral ventral midbrain, and degeneration of dopaminergic projection in the ipsilateral striatum (data not shown). Three weeks after 6-OHDA infusion, AAV-GFAP-CasRx-Ptbp1 (or AAV-GFAP-CasRx as a control) together with AAV-GFAP-mCherry were injected into the ipsilateral striatum. Analysis of striatal cell types was performed at different time points after AAV injection (FIGS. 16 and 17). Interestingly, 6-OHDA-lesioned mice injected with AAV-GFAP-CasRx-Ptbp1 and AAV-GFAP-mCherry showed a high percentage of cells expressing both TH and mCherry at one month after injection, and the percentage increased at three months (19±0.4%, SEM, n=5 mice, at 1 month; 32±7%, SEM, n=3 mice, at 3 months) (FIGS. 18 and 19). Such cells were rarely observed in mice at one and two week after injection or injected with control AAVs (FIGS. 18 and 19). In addition, around 80% of TH⁺ cells in the virus-injected region were mCherry⁺ (FIG. 20), suggesting that they were mainly derived from astrocytes. The percentage of mCherry⁺ TH⁺ cells in mCherry⁺ cells in wild-type (non-PD without 6-OHDA lesion) mice was lower than that of 6-OHDA lesioned mice (FIG. 21), suggesting that endogenous repair mechanisms may promote the induction of dopamine neurons after injury.

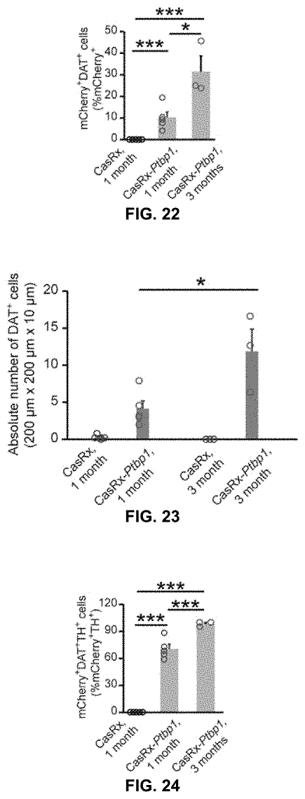

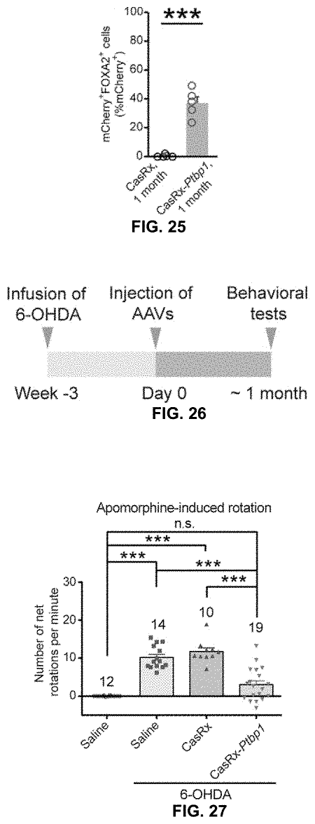

Induced neurons expressed the mature dopamine neuron marker dopamine transporter Slc6a3 (DAT), which is present in midbrain dopamine neurons but absent in the lesion-induced transiently TH-expressing striatal neurons. A high percentage (10±3%, SEM, n=5 mice, at 1 month; 31±7%, SEM, n=3 mice, at 3 months) of mCherry⁺ DAT⁺ cells was found in the AAV-GFAP-CasRx-Ptbp1-injected striatum, but not in mice injected with the control AAV (FIGS. 22 and 23). Further co-immunostaining of TH and DAT revealed that the majority of mCherry⁺ TH⁺ cells expressed DAT (FIG. 24), indicating that most dopamine neurons converted from astrocytes were mature. In addition, mCherry⁺ cells expressed two other midbrain dopamine neuron markers, DOPA-decarboxylase (DDC) and forkhead box protein A2 (FOXA2) (FIG. 25), further confirming that these are astrocyte-derived dopaminergic neurons.

Previous studies reported the presence of TH⁺ interneurons in the mouse striatum after 6-OHDA lesion. Here, the appearance of induced TH⁺ interneurons was evaluated and shown by PV⁺, SST⁺ and Calretinin⁺ (CR⁺) cells with mCherry expression at 3 months after AAV injection and found that none of these interneuron markers colocalized with mCherry⁺ TH⁺ cells, suggesting that converted TH⁺ dopamine neurons were not transiently induced TH⁺ interneurons (data not shown).

To explore the subtype identity of induced dopamine neurons, TH were co-immunostained with two SNc A9 area-specific dopamine neuron markers ALDH1A1 and GIRK2, respectively, and DAT with a ventral tegmental area (VTA)-specific dopamine neuron marker Calbindin. The results showed that almost all induced dopamine neurons expressed ALDH1A1 and GIRK2 but not Calbindin (data not shown), suggesting that induced dopamine neurons shared many characteristics with SNc dopamine neurons.

Whole-cell recording was also performed on striatal slices of injected mice. The majority of neuron-like mCherry⁺ cells (20 out of 22 cells) were capable of generating repetitive action potentials in response to depolarizing current injection in the current-clamp mode (data not shown). Spontaneous postsynaptic currents were also observed in the voltage-clamp mode (Vc=−70 mV), indicating that converted neurons received functional synaptic inputs (data not shown). Moreover, in 4 out of 10 neurons examined, delayed voltage rectification (data not shown) induced by hyperpolarization-activated currents (Ih), a signature of midbrain dopamine neurons (Engel, 2016), was observed.

The induced dopamine neurons could also release dopamine. The majority of mCherry⁺ TH⁺ cells expressed vesicular monoamine transporter 2 (VMAT2) (data not shown), an essential protein that regulates the packaging, storage and release of dopamine. Many cells in the virus-injected striatum region showed uptake of a fluorescent dopamine derivative (FFN206), an VMAT2 substrate that is able to detect active VMAT2 in intact cells, and partial reduction of the fluorescence upon high KCl treatment, suggesting the capability of dopamine release function of the converted cells (data not shown). Based on the expression of VMAT2 in the soma, and the reduction of FFN206 in the soma after KCl treatment, it was speculate that release of dopamine from soma is the most likely mechanism, although release from neurites could not be excluded.

Taken together, the results showed that CasRx-mediated Ptbp1 knockdown could efficiently convert striatal astrocytes into functional dopamine neurons in the striatum of PD model mice.

### Example 6 Astrocyte-to-Neuron Conversion Alleviated Motor Dysfunctions in PD Mice

This example demonstrates that conversion of astrocytes into dopamine neurons in the striatum alleviated the symptoms in the 6-OHDA-induced PD mouse model (FIG. 26).

The motor functions were evaluated for drug-induced and drug-free activities.

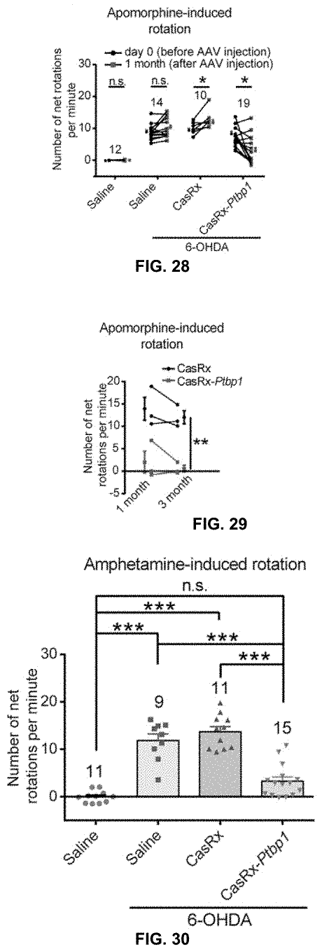

For drug-induced activities, apomorphine-induced contralateral rotation behavior, which is widely used for demonstrating unilateral dopamine neuronal loss, was first examined. Apomorphine-induced net rotation (counted as contralateral-ipsilateral rotation number) was significantly diminished in Ptbp1-knockdown mice injected with AAV-GFAP-CasRx-Ptbp1 and AAV-GFAP-mCherry, as compared to control mice injected with AAV-GFAP-CasRx and AAV-GFAP-mCherry, or with saline, to the level comparable to that found in non-lesioned wide-type mice (FIGS. 27-29).

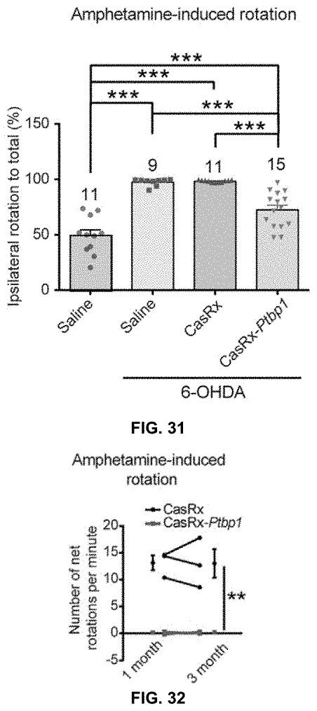

Another ipsilateral preferred rotation behavior induced by systemic amphetamine administration, which increases intercellular dopamine concentration by inhibiting dopamine re-uptake of DAT in the striatum, also showed marked reduction of net rotation (counted as ipsilateral-contralateral rotation number) and ipsilateral rotation ratio (counted as ipsilateral/total rotation number) in mice injected with AAV-GFAP-CasRx-Ptbp1, as compared to control mice (FIGS. 30-32).

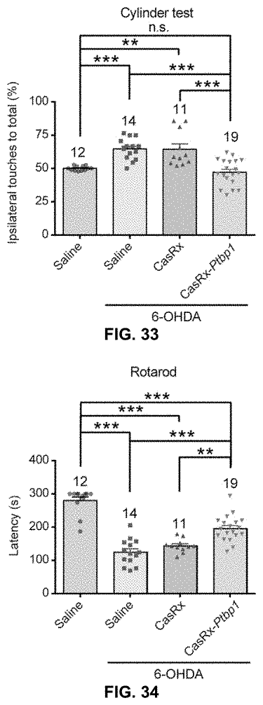

These results suggest that astrocyte-derived dopamine neurons in the striatum could release sufficient dopamine to reduce motor dysfunction revealed by the drug-induced rotation behavior in the PD model mice. In addition, two drug-free motor dysfunctions, the forelimb-use asymmetry and motor coordination, were examined using cylinder and rotarod tests, respectively. Mice injected with AAV-GFAP-CasRx-Ptbp1 showed significantly lower percentages of ipsilateral touches of the cylinder and longer duration on the rotarod, as compared to control mice (FIGS. 33 and 34).

Together, these results showed that astrocyte-to-dopamine neuron conversion due to Ptbp1 knockdown in the striatum alleviated the motor dysfunctions in the PD mouse model.

### Example 7 Detection of Off-Target Ptbp1 Knockdown Tools In Vitro

In order to explore whether the subsequent Cas13Rx and Cas13e can be applied to clinical in vivo treatment, the present invention attempts to use an in vitro system to perform off-target detection of these RNA editing tools in the process of knocking down Ptbp1. Firstly, a plasmid containing CasRx-sgRNA (the sgRNA combination is from Cell, DOI: 10.1016/j.cell.2020.03.024) and Cas13e-sgRNA (the sgRNA combination is a newly screened combination, which is shown as follows: sgRNA1:

(the sgRNA combination was a newly
screened combination,
sgRNA1: TGTGGTTGGAGAACTGGATGTAGATGGGCT
(SEQ ID NO. 7),
sgRNA2: GAGCCCATCTGGATCAGTGCCATCTTGCGG
(SEQ ID NO.: 8);
sgRNA3: AGTCGATGCGCAGCGTGCAGCAGGCGTTGT
(SEQ ID NO.: 9))

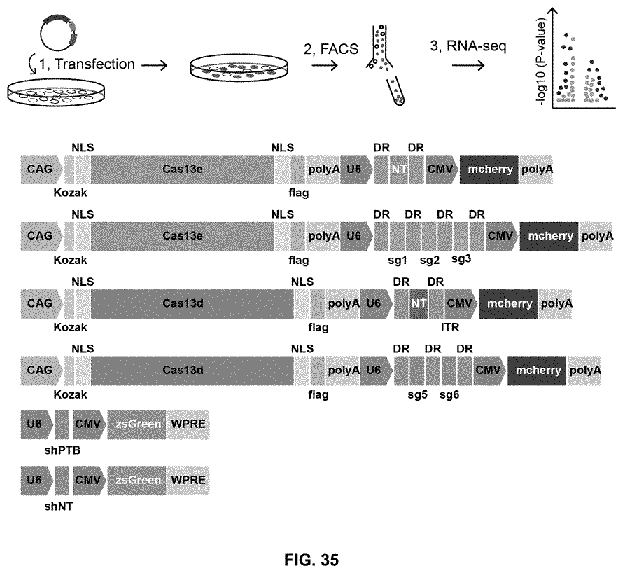

and a plasmid containing Cas13e-NT, CasRx-NT, U6-shPTB (shPTB is from Nature, DOI: 10.1038/s41586-020-2388-4) and U6-shNT were constructed and used as controls (FIG. 35). In addition, they were transferred into N2a cells by liposome transfection. Since these vector plasmids carry the mcherry reporter gene, 48 hours after transfection, we used flow sorting to sort out mcherry-positive cells and collected about 50,000 cells in each group and RNA whole transcriptome sequencing was performed. By analyzing the results of RNA whole transcriptome sequencing, it can be found that the off-target rate of Cas13e-sgRNA is lower than that of CasRx-sgRNA, and the off-target rate of Cas13e-sgRNA and CasRx-sgRNA is much lower than that of U6-shPTB group. The results show that CRISPR-mediated RNA editing tools, especially Cas13e, are superior to traditional shRNA tools in off-target effects.

### Example 10 Detection of Transdifferentiation Efficiency after Knockdown of Ptbp1 In Vivo

In order to further explore the potential use of Cas13e in the process of inducing glial cells to differentiate into neurons, we examined whether Cas13e knocking down Ptbp1 in astrocytes in the striatum can convert it into dopamine neurons and compared it with the two tools CasRx and shRNA. For this reason, a Cas13e-sgRNA plasmid driven by GFAP promoter was constructed

(the sgRNA combination was a newly
screened combination,
sgRNA1: TGTGGTTGGAGAACTGGATGTAGATGGGCT
(SEQ ID NO. 7),
sgRNA2: GAGCCCATCTGGATCAGTGCCATCTTGCGG
(SEQ ID NO.: 8;
sgRNA3: AGTCGATGCGCAGCGTGCAGCAGGCGTTGT
(SEQ ID NO.: 9)),

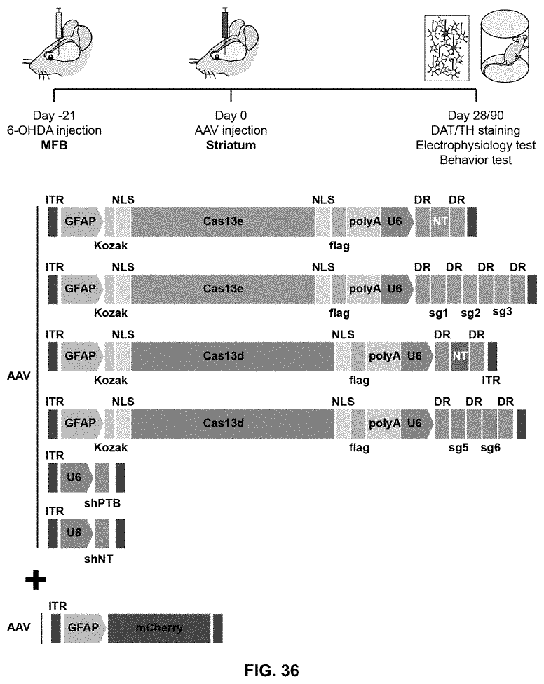

and a CasRx-sgRNA plasmid driven by GFAP promoter was constructed (the sgRNA combination was from Cell, DOI: 10.1016/j.cell.2020.03.024) and a plasmid containing Cas13e-NT, CasRx-NT, U6-shPTB (shPTB is from Nature, DOI: 10.1038/s41586-020-2388-4) and U6-shNT were constructed and they were used as controls (FIG. 36). These plasmids were then packaged and purified by adeno-associated virus AAV. Three weeks before the virus injection experiment, we infused 6-hydroxydopamine (6-OHDA) unilaterally into the substantia nigra to induce a Parkinsonian mouse model, and then these AAV viruses were injected into the striatum of these Parkinson mice. In addition, AAV-GFAP-mCherry virus was injected to label astrocytes (FIG. 36). 28 days and 90 days after virus injection, DAT/TH staining, electrophysiological and mouse behavior tests were performed respectively to verify the transdifferentiation efficiency in vivo from various aspects. We have found that the combination of Cas13e-sgRNA can also effectively knock down Ptbp1 in mice, and lead to the differentiation of astrocytes into dopamine neurons, and improve the motor function of Parkinson's disease model mice, it also has potential prospects in clinical treatment.

1. A method of generating a functional RGC (retinal ganglion cell) in an eye of a mammalian subject, comprising
1) suppressing an expression or activity of a PTB (Polypyrimidine Tract-Binding Protein) in a glial cell in a mature retina of the mammalian subject;
2) allowing said glial cell to reprogram; and
3) generating the functional RGC by the reprogramed glial cell.

2. The method of claim 1, wherein suppressing the expression or activity of the PTB comprises expressing in said glial cell a CRISPR/Cas effector protein and a guide RNA (gRNA) complementary to a PTB mRNA.

3. The method of claim 2, wherein said Cas effector protein is selected from the group consisting of Cas13d, CasRx, Cas13e, Cas13f, CRISPR/Cas9, Cpf1, Cas9, Cas13a, Cas13b, Cas13c, and a combination thereof.

4. The method of claim 2, wherein said CRISPR/Cas effector protein and/or said gRNA are encoded by an expression vector, and under the transcriptional control of a glial cell-specific promoter.

5. The method of claim 4, wherein the expression vector comprises an AAV vector, wherein the AAV vector encodes both the CRISPR/Cas effector protein and the gRNA, each specific for a different target region of the PTB mRNA.

6. The method of claim 4, wherein the AAV vector is an AAV2 vector, or an AAV9 vector.

7. The method of claim 1, wherein said glial cell is an Muller glia cell.

8. The method of claim 1, wherein said RGC comprises a RGC
(1) expressing Brn3a, Rbpms, Foxp2, Brn3c, or Parvalbumin;
(2) being F-RGC, RGC subtype 3, or PV-RGC;
(3) being integrated in existing retinal circuitry in said mammalian subject; or
(4) capable of receiving visual information characterized by its ability to establish action potential upon light stimulation, synaptic connections, biogenesis of pre-synaptic neurotransmitter, and/or post-synaptic response.

9. The method of claim 1, wherein the method reprograms a plurality of glial cells in said mature retina, and wherein at least 10% of said glial cells are converted to RGCs.

10. The method of claim 1, wherein said mammalian subject is a human, or a non-human animal.

11. The method of claim 10,
wherein the mammalian subject is human, and
wherein the method further comprises after step 1) and before step 2)
1a) allowing an nPTB (polypyrimidine tract binding protein 2) in the glial cell to express to a high nPTB expression level; and
1b) suppressing the expression or activity of the nPTB in the glial cell.

12. The method of claim 11, wherein said high nPTB expression level is a level achieved about 3 days, about 1 week, about 10 days, about 2 weeks, about 3 weeks, or about 4 weeks after suppressing the expression or activity of the PTB.

13. The method of claim 11, wherein suppressing the expression or activity of the nPTB comprises expressing in said glial cell a CRISPR/Cas effector protein and a guide RNA (gRNA) complementary to a nPTB mRNA.

14. A method of treating a neurological condition associated with degenerated functional neurons in a mature retina of a subject in need thereof, comprising
1) suppressing an expression or activity of a PTB in a glial cell in the mature retina of the subject;
2) allowing said glial cell to reprogram into a functional neuron in the mature retina; and
3) replenishing said degenerated functional neurons in said mature retina with the functional neuron, thereby treating said neurological condition.

15. The method of claim 14, wherein said neurological condition is selected from the group consisting of glaucoma, age-related RGC loss, optic nerve injury, retinal ischemia, and Leber's hereditary optic neuropathy.

16. A method of treating a neurological condition associated with degenerated RGC neurons, comprising
1) suppressing an expression or activity of a PTB in a glial cell in a mature retina of a subject;
2) allowing said glial cell to reprogram into a RGC neuron; and
3) replenishing said degenerated RGC neurons in said mature retina with the RGC neuron, thereby treating said neurological condition.

18-37. (canceled)

38. A composition comprising
1) a CRISPR/Cas effector protein or an expression vector encoding a CRISPR/Cas effector protein; and
2) a guide RNA (gRNA) complementary to a PTB mRNA or an expression vector encoding a guide RNA (gRNA) complementary to a PTB mRNA;
wherein the composition when administered to a mammalian subject is capable of generating the functional RGC in the eye of the mammalian subject as defined in the method of claim 1.

39. The composition of claim 38, wherein the composition is formulated for injection, inhalation, parenteral administration, intravenous administration, subcutaneous administration, intramuscular administration, intradermal administration, topical administration, or oral administration.

40. The composition of claim 38,
wherein the composition is an injectable composition; and
wherein the expression vector encoding a CRISPR/Cas construct is configured to suppress an expression or activity of a PTB in a glial cell.

41. The injectable composition of claim 40, wherein said glial cell is an astrocyte, an oligodendrocyte, an ependymal cell, a Schwan cell, a NG2 cell, or a satellite cell.

42. (canceled)


SEQUENCE LISTING (9 sequences)

SEQ ID NO: 1 - DNA, 29 bp, artificial sequence
  Guide RNA sequence
tttgtaccgactgctatgtctgggacgat

SEQ ID NO: 2 - DNA, 30 bp, artificial sequence
  Guide RNA sequence
ggctggctgtctccagagggcaggtcaggt

SEQ ID NO: 3 - DNA, 30 bp, artificial sequence
  Guide RNA sequence
gtatagtagttaaccatagtgttggcagcc

SEQ ID NO: 4 - DNA, 30 bp, artificial sequence
  Guide RNA sequence
gctgtcggtcttgagctctttgtggttgga

SEQ ID NO: 5 - DNA, 30 bp, artificial sequence
  Guide RNA sequence
tgtagatgggctgtccacgaagcactggcg

SEQ ID NO: 6 - DNA, 30 bp, artificial sequence
  Guide RNA sequence
gcttggagaagtcgatgcgcagcgtgcagc

SEQ ID NO: 7 - DNA, 30 bp, artificial sequence
  sgRNA1
tgtggttggagaactggatgtagatgggct

SEQ ID NO: 8 - DNA, 30 bp, artificial sequence
  sgRNA2
gagcccatctggatcagtgccatcttgcgg

SEQ ID NO: 9 - DNA, 30 bp, artificial sequence
  sgRNA3
agtcgatgcgcagcgtgcagcaggcgttgt